(19) United States
(12) Patent Application Publication
(10) Pub. No.: US 2025/0368757 A1
(43) Pub. Date: Dec. 4, 2025

(54) PARTICLES COMPRISING PROTEINS ENCAPSULATED IN A POROUS FRAMEWORK AND METHODS OF USING THEREOF

(71) Applicant: Ohio State Innovation Foundation, Columbus, OH (US)

(72) Inventors: Xiangming GU, Columbus, OH (US); Andre PALMER, Columbus, OH (US)

(21) Appl. No.: 18/854,305

(22) PCT Filed: Apr. 4, 2023
(86) PCT No.: PCT/US23/17422

§ 371 Date: Oct. 4, 2024

Related U.S. Application Data
(60) Provisional application No. 63/327,126, filed on Apr. 4, 2022.
(60) Provisional application No. 63/387,204, filed on Dec. 13, 2022.

(51) Int. Cl. C07K 17/14; A61K 9/51; A61K 38/42; C07K 17/04

(52) U.S. Cl. CPC C07K 17/14; A61K 9/5123; A61K 9/5146; A61K 38/42; C07K 17/04

(57) ABSTRACT

Disclosed are methods for producing matrix-encapsulated proteins, including matrix-encapsulated hemoglobin. Also provided are pharmaceutical compositions comprising a matrix-encapsulated hemoglobin, as well as methods of using thereof to treat hypoxia, cyanide poisoning, hydrogen sulfide poisoning, and/or azide poisoning.

### CROSS-REFERENCE TO RELATED APPLICATIONS

This application claims benefit of U.S. Provisional Application No. 63/327,126, filed Apr. 4, 2022, and U.S. Provisional Application No. 63/387,204, filed Dec. 13, 2022, each of which is hereby incorporated herein by reference in its entirety.

### STATEMENT REGARDING FEDERALLY SPONSORED RESEARCH OR DEVELOPMENT

This invention was made with government support under grant numbers R01HL126945, R01HL138116, R01HL156526, and R01EB021926 awarded by the National Institutes of Health. The government has certain rights in the invention.

### BACKGROUND

Hemoglobin (Hb)-based oxygen carriers (HBOCs) constitute one major class of artificial red blood cell (RBC) substitute, which has gained considerable attention in treating hemorrhagic shock, ameliorating traumatic brain injury, and suppressing tumor growth via oxygenation of hypoxic tissues. Unfortunately, prior generations of HBOCs yielded disappointing clinical outcomes due to significant safety concerns. Extravasation of cell-free Hb initially present in the vascular space through the vascular endothelium into the tissue space is regarded as the primary underlying cause of those safety concerns, which manifests through vasoconstriction, systemic hypertension, and oxidative tissue injury.

Fortunately, various strategies have been developed to reduce/prevent extravasation of cell-free Hb including Hb polymerization, surface conjugation of Hb, and liposome encapsulation of Hb. All of these strategies increase the molecular radius of Hb, so that it is unable to extravasate through the blood vessel wall. It was observed that chemical modification of Hb via polymerization and surface conjugation approaches drastically decrease the flexibility of Hb, which affects cooperative oxygen (O₂) binding and release. Although liposome encapsulation had a negligible impact on cooperative O₂ binding/release, low Hb encapsulation efficiency remains a significant issue that impeded scale-up and potential commercialization. Those underlying flaws associated with liposome encapsulated Hb nanoparticles underscore the need to develop next generation Hb nanoparticles with enhanced uniformity, high Hb content, and high encapsulation efficiency without jeopardizing cooperative O₂ binding and release. Further, strategies developed to produce encapsulated Hb particles should also provide access to a variety of other encapsulated proteins.

### SUMMARY

Provided herein are method for producing a population of matrix-encapsulated protein particles. These methods can comprise: (a) combining a first framework precursor, a second framework precursor, and a protein to form a reactant mixture; (b) incubating the reactant mixture under conditions effective to form the population of matrix-encapsulated protein particles; and (c) separating the matrix-encapsulated protein from the reactant mixture using ultrafiltration. The resulting matrix-encapsulated protein particles can comprise a protein encapsulated in a porous framework formed by reaction of the first framework precursor and the second framework precursor.

In some embodiments, step (c) comprises filtering the reactant mixture comprising the matrix-encapsulated protein particles by ultrafiltration against a filtration membrane, thereby forming a retentate fraction comprising matrix-encapsulated protein particles having a molecular weight above a cutoff value and a permeate fraction comprising unencapsulated protein and other impurities having a molecular weight of less than the cutoff value.

The cutoff value can be between the molecular weight of the protein present in the reaction mixture and the average particle size of the matrix-encapsulated protein particles (i.e., so as to facilitate efficient separation of the matrix-encapsulated protein particles from unencapsulated protein and other impurities remaining in the reactant mixture). In some embodiments, the cutoff value is from 50 kDa to 1000 kDa (e.g., from 150 kDa to 750 kDa, from 250 to 750 kDa, or from 400 kDa to 600 kDa). In certain embodiments, the filtration membrane can be rated for retaining solutes having a molecular weight of greater than 50 kDa, such as greater than 100 kDa, greater than 150 kDa, greater than 250 kDa, greater than 300 kDa, or greater than 500 kDa.

In certain embodiments, the ultrafiltration can comprise tangential flow filtration or cross-flow filtration.

The porous framework can comprise, for example, a metal-organic framework (MOF), metal-inorganic framework (MIF), and/or covalent-organic framework (COF). In certain embodiments, the porous framework can comprise a MOF.

In some embodiments, the first framework precursor can comprise a metal salt and the second framework precursor can comprise a ligand. For example, in some examples, the first framework precursor can comprise a Fe salt, a Co salt, a Cu salt, a Zn salt, or a combination thereof. In some examples, the second precursor can comprise a ligand selected from the group consisting of imidazoles and derivatives such as 2-methylimidazole, 2-ethylimidazole, 4-azabenzimidazole, benzimidazole, nitroimidazole, 2-chloroimidazole, and the like; carboxylic acids and derivatives such as 1,4-benzenedicarboxylic acid, 1,3,5-benzene tricarboxylic acid, imidazole carboxaldehyde, 2-aminobenzimidazolate, the like, or any combination thereof.

In some embodiments, the first framework precursor and the second framework precursor can be present in the reactant mixture at a molar ratio of from 1:1 to 75:1, such as from 1:1 to 60:1, from 1:1 to 30:1, or from 15:1 to 30:1.

In certain embodiments, the porous framework can comprise a zeolitic imidazolate framework (ZIF). For example, the porous framework can comprise a zeolitic imidazolate framework such as ZIF-2, ZIF-3, ZIF-4, ZIF-8, ZIF-9, ZIF-10, ZIF-11, ZIF-12, ZIF-14, ZIF-20, ZIF-21, ZIF-23, ZIF-60, ZIF-61, ZIF-62, ZIF-64, ZIF-65, ZIF-67, ZIF-68, ZIF-69, ZIF-70, ZIF-71, ZIF-72, ZIF-73, ZIF-74, ZIF-75, ZIF-76, ZIF-77, ZIF-90, derivatives thereof, and combinations thereof.

The protein can comprise any suitable protein. For example, the protein can comprise conalbumin, albumin, hemoglobin, haptoglobin, hemopexin, transferrin, methemoglobin, ovalbumin, a-chymotrypsinogen A, a-chymotrypsin, trypsin, trypsinogen, B-lactoglobulin, myoglobin, a-lactalbumin, lysozyme, ribonuclease A, or cytochrome c, a recombinant version thereof, or a combination thereof. In some embodiments, the protein can be surface conjugated. In some embodiments, the protein can comprise a globular protein. In certain embodiments, the protein can comprise hemoglobin. The hemoglobin can be from a mammalian, invertebrate, or recombinant source. For example, the hemoglobin can comprise human hemoglobin, bovine hemoglobin, or porcine hemoglobin. In some embodiments, the hemoglobin can comprise a polymerized hemoglobin. The polymerized hemoglobin can be in the tense or relaxed quaternary state, or is in between these two quaternary states.

In some embodiments, the reactant mixture can further comprise an etching agent, a chelating agent, or a combination thereof. For example, the etching agent can comprise hydrofluoric acid (HF), ammonium fluoride (NH₄F), the acid salt of ammonium fluoride (NH₄HF₂), sodium hydroxide (NaOH), nitric acid (HNO₃), hydrochloric acid (HCl), hydroiodic acid (HI), hydrobromic acid (HBr), boron trifluoride (BF₃), sulfuric acid (H₂SO₄), acetic acid (CH₃COOH), formic acid (HCOOH), phosphoric acid (H₃PO₄), or any combination thereof. The chelating agent can comprise, for example, ethylenediaminetetraacetic acid (EDTA) or a derivative thereof.

The resulting population of matrix-encapsulated protein particles can have any suitable size. In some cases, the population of matrix-encapsulated protein particles can comprise microparticles. In other embodiments, the population of matrix-encapsulated protein particles can comprise nanoparticles. In certain embodiments, the population of matrix-encapsulated protein particles can have an average particle size, as determined by electron microscopy, of less than 200 nm, such as less than 180 nm, less than 160 nm, less than 140 nm, less than 120 nm, less than 100 nm, or less than 80 nm.

In some embodiments, the population of matrix-encapsulated protein particles can have a PDI of less than 0.100, such as less than 0.095, such as less than 0.090, less than 0.085, less than 0.080, less than 0.075, or less than 0.070.

In some embodiments, the population of matrix-encapsulated protein particles can have a zeta potential of less than −5 mV, such as of less than −6 mV, less than −7 m V, less than −8 mV, less than −9 mV, less than −10 mV, less than −11 mV, less than −12 mV, less than −13 mV, less than −14 m V, or less than −15 mV.

In some embodiments, the protein can retain its biological activity following encapsulation. For example, in some embodiments, at least 90% of the of the protein in the population of matrix-encapsulated protein particles retain their biological activity. For example, in the case of methods related to the encapsulation of hemoglobin, in some embodiments, at least 90% of the of the protein in the population of matrix-encapsulated protein particles comprises hemoglobin (and less than 10% of the protein comprises methemoglobin).

In some embodiments, the methods can encapsulate a protein with relatively high encapsulation efficiency. For example, in some embodiments, the method can encapsulate a protein with an encapsulation efficiency, measured by the fraction of the mass of the protein in the resulting matrix-encapsulated protein over the total mass of protein initially charged in the reactant mixture, of at least 80%, such as at least 82%, at least 84%, at least 86%, at least 88%, at least 90%, at least 92%, at least 94%, or at least 96%.

Also provided are pharmaceutical compositions comprising a population of matrix-encapsulated hemoglobin particles prepared using the methods described herein. The compositions can be administered to a subject in need thereof, for example, to treat hypoxia (e.g., hypoxia is at least partially caused by traumatic brain injury or hemorrhagic shock).

The details of one or more embodiments of the disclosure are set forth in the accompanying drawings and the description below. Other features, objects, and advantages of the disclosure will be apparent from the description and drawings, and from the claims.

### DESCRIPTION OF DRAWINGS

FIG. 1A is a schematic of ZIF-8P-Hb nanoparticle formation pathway induced by the coordination of Hmim and Zn.

FIG. 1B is a schematic illustration of the synthesis and purification of ZIF-8P-Hb nanoparticles starting from bHb precursor.

FIGS. 2A-2D show the results of the PXRD analysis and Cryo-EM of ZIF-8 and ZIF-8P-Hb nanoparticles. FIG. 2A is an image of Hb, ZIF-8 and ZIF-8P-Hb nanoparticle solutions. A clear color difference was observed between ZIF-8 and ZIF-8P-Hb nanoparticle suspensions, demonstrating successful bHb encapsulation. FIG. 2B shows the diffraction patterns of ZIF-8, ZIF-8P-Hb5 (Hbᵢₙᵢₜᵢₐₗ=5 mg/mL), and ZIF-8P-Hb 10 (Hbᵢₙᵢₜᵢₐₗ=10 mg/mL) nanoparticles. FIG. 2C shows Cryo-EM images of ZIF-8. FIG. 2D shows Cryo-EM images of ZIF-8P-Hb5.

FIG. 3 is a plot showing the zeta potential of ZIF-8 and ZIF-8P-Hb nanoparticles in comparison to previously reported ZIF-8 encapsulated Hb nanoparticles including ZIF-8@Hb-1, ZIF-8@Hb-2, ZIF-8@Hb-5, and ZIF-8@Hb-10.

FIGS. 4A-4F show meta-data analysis of procedural parameters for ZIF-8 and ZIF-8P-Hb nanoparticle synthesis. FIG. 4A shows the effect of the initial concentration of bHb on the hydrodynamic diameter of ZIF-8P-Hb nanoparticles. FIG. 4B shows the etching effect of EDTA on the hydrodynamic diameter of ZIF-8 nanoparticles. FIG. 4C shows the aggregation effect of pump flow rate during the TFF particle washing process on the hydrodynamic diameter of ZIF-8 nanoparticles. FIG. 4D shows the effect of zinc nitrate hexahydrate concentration on the hydrodynamic diameter of ZIF-8 nanoparticles. FIG. 4E shows the effect of the molar ratio of Hmim:Zn on the hydrodynamic diameter of ZIF-8P-Hb nanoparticles. FIG. 4F shows the effect of different addition methods on the hydrodynamic diameter of ZIF-8 nanoparticles.

Figure SA shows the O₂ equilibrium curves for bHb and ZIF-8P-Hb nanoparticles in comparison to bHb, RBCs, and various types of HBOCs in the literature. Lines represent the mean from all batches.

FIG. 5B shows the normalized deoxygenation kinetics of bHb, ZIF-8P-Hb nanoparticles, and several HBOCs from the literature. The absorbance was monitored at 437.5 nm and normalized against the maximum value.

FIG. 5C shows the pseudo first order haptoglobin (Hp) binding kinetics. The normalized fluorescence changes were fit to a monoexponential equation.

FIG. 5D shows the second order Hp binding kinetics. The second order Hp binding rate constants were obtained by performing a linear fit of the pseudo first order Hp binding rate constants as a function of bHb concentration.

FIG. 6A shows ZIF-8P-Hb hydrothermal stability evaluated by measuring bHb release from the ZIF-8P-Hb nanoparticle suspension at 4° C. over 14 days. The mass of bHb released from the ZIF-8P-Hb nanoparticles during the storage study was defined as the difference between the initial mass of bHb released from the ZIF-8P-Hb nanoparticles and the mass of bHb released after day 1. The mass of released bHb was calculated by measuring the bHb concentration and volume of the permeate solution collected from a 500 kDa TFF membrane.

FIG. 6B shows the hemocompatibility of ZIF-8P-Hb nanoparticles evaluated via a hemolysis assay as previously described in the literature. Hb release from the RBCs was measured by UV-visible spectroscopy after exposure to ZIF-8P-Hb nanoparticles (0.6 mM, heme basis). C1, C2, C3, and C4 correspond to the Hb concentration in the supernatant of the unlysed RBC sample, fully lysed RBC sample, ZIF-8P-Hb nanoparticles alone, and ZIF-8P-Hb nanoparticles/RBC mixture, respectively. Given that all samples were diluted to an equal volume of 2 mL with 0.9% saline, the extent of hemolysis can be calculated using the Hb concentration.

FIG. 7A shows a procedural timeline for T-state PolybHb synthesis and purification. FIG. 7B is a schematic description of the reaction pathway for ZIF-8P-PolybHb NPs. FIG. 7C illustrates the synthesis and purification of ZIF-8P-PolybHb NPs.

FIGS. 8A-8G illustrate the morphology and structural analysis of ZIF-8, ZIF-8P-Hb, and ZIF-8P-PolybHb NPs. FIG. 8A shows images of ZIF-8, ZIF-8P-Hb, PolybHb, and ZIF-8P-PolybHb NP solutions. FIG. 8B shows the diffraction patterns of ZIF-8, ZIF-8P-Hb, and ZIF-8P-PolybHb NPs. FIG. 8C-8E show TEM images of ZIF-8 NPs (FIG. 8C), ZIF-8P-Hb NPs (FIG. 8D), and ZIF-8P-PolybHb NPs (FIG. 8E). FIGS. 8F and 8G show the zeta potential (FIG. 8F) and hydrodynamic diameter (FIG. 8G) of ZIF-8P-Hb NPs, PolybHb, and ZIF-8P-PolybHb NPs in comparison to bare ZIF-8 NPs.

FIG. 9A shows the O₂ equilibrium curve of ZIF-8P-PolybHb NPs in comparison to bHb, PolybHb, ZIF-8P-Hb NPs, RBCs, and HbV. Lines represent the mean from all batches. The standard deviation is represented by the shaded area.

FIG. 9B shows the normalized deoxygenation kinetics of ZIF-8P-PolybHb NPs, PolybHb, ZIF-8P-Hb NPs, HbVs, RBCs, and bHb. The absorbance was monitored at 437.5 nm and normalized against the maximum value.

FIG. 9C shows the pseudo first order haptoglobin (Hp) binding kinetics. The normalized fluorescence changes were fit to a monoexponential equation.

FIG. 9D show the second order Hp binding kinetics. The second order Hp binding rate constants were obtained by performing a linear fit of the pseudo first order Hp binding rate constants as a function of Hb concentration.

FIG. 10A shows the auto-oxidation kinetics of bHb, PolybHb, and ZIF-8P-PolybHb NPs. The auto-oxidation rate was assessed by monitoring the metHb level of bHb, PolybHb, and ZIF-8P-PolybHb solutions at 37° C. over 24 hours. The metHb level was measured via the cyanmethemoglobin method. First order auto-oxidation rate constants were calculated by performing a linear regression on the natural log of the normalized concentration of the materials on a heme-basis as a function of time. FIG. 10B shows the antioxidant properties of Hb, PolybHb, ZIF-8P-Hb, and ZIF-8P-PolybHb NPs were studied via incubating the samples with an excessive amount of H₂O₂ (100 mM). After exposure to H₂O₂ for 5 mins at room temperature, the UV-visible spectra of bHb, PolybHb, ZIF-8P-Hb, and ZIF-8P-PolybHb NPs at a concentration of 0.125 mg/mL, 0.25 mg/mL, and 0.5 mg/mL were collected. Spectral deconvolution analysis was then performed to assess the concentrations of oxyHb, metHb, and hemichrome species (* denotes a statistically significant difference (p<0.05) when compared to bHb; † denotes a statistically significant difference (p<0.05) when compared to PolybHb; # denotes a statistically significant difference (p<0.05) when compared to ZIF-8P-Hb NPs; † denotes statistically significant difference (p<0.05) when compared to ZIF-8P-PolybHb).

FIG. 11A shows that ZIF-8P-PolyHb NP hydrolytic stability was studied by monitoring PolybHb release from the ZIF-8P-PolybHb NP solution at 37° C. over 7 days. The mass of PolybHb released from the ZIF-8P-PolybHb NPs was measured by collecting the total volume of the permeate solution from a 500 kDa TFF membrane and measuring the particle-free PolybHb concentration. FIG. 11B shows a SEC-HPLC chromatogram of released PolybHb from ZIF-8P-PolyHb NPs after 24 hours at 37° C.

FIG. 12 is a plot showing the in vitro cytotoxicity of ZIF-8P-PolybHb NPs, measured in HUVECs, showing no statistically significant decrease in cell viability at all tested concentrations compared to the untreated positive control. Unencapsulated PolybHb also showed negligible impact on cell viability, but encapsulation of PolybHb into ZIF-8 NPs improved NP cytotoxicity compared to unloaded ZIF-8 NPs and ZIF-8-bHb NPs that showed statistically significant differences compared to the positive control (p<0.05). * denotes statistical significance compared to the positive control (i.e., untreated cells in cell culture media).

FIG. 13 illustrates the encapsulation of polymerized hemoglobin in the tense quaternary state inside a nanoparticle comprised of zeolite imidazole framework precursors with antioxidant properties.

### DETAILED DESCRIPTION

A number of embodiments of the disclosure have been described. Nevertheless, it will be understood that various modifications may be made without departing from the spirit and scope of the invention. Accordingly, other embodiments are within the scope of the following claims.

### Definitions

To facilitate understanding of the disclosure set forth herein, a number of terms are defined below. Unless defined otherwise, all technical and scientific terms used herein generally have the same meaning as commonly understood by one of ordinary skill in the art to which this disclosure belongs. Publications cited herein and the materials for which they are cited are specifically incorporated by reference.

### General Definitions

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than where noted, all numbers expressing quantities of ingredients, reaction conditions, geometries, dimensions, and so forth used in the specification and claims are to be understood at the very least, and not as an attempt to limit the application of the doctrine of equivalents to the scope of the claims, to be construed in light of the number of significant digits and ordinary rounding approaches.

As used in this specification and the following claims, the terms “comprise” (as well as forms, derivatives, or variations thereof, such as “comprising” and “comprises”) and “include” (as well as forms, derivatives, or variations thereof, such as “including” and “includes”) are inclusive (i.e., open-ended) and do not exclude additional elements or steps. For example, the terms “comprise” and/or “comprising,” when used in this specification, specify the presence of stated features, integers, steps, operations, elements, and/or components, but do not preclude the presence or addition of one or more other features, integers, steps, operations, elements, components, and/or groups thereof. Accordingly, these terms are intended to not only cover the recited element(s) or step(s), but may also include other elements or steps not expressly recited. Furthermore, as used herein, the use of the terms “a”, “an”, and “the” when used in conjunction with an element may mean “one,” but it is also consistent with the meaning of “one or more,” “at least one,” and “one or more than one.” Therefore, an element preceded by “a” or “an” does not, without more constraints, preclude the existence of additional identical elements.

The use of the term “about” applies to all numeric values, whether or not explicitly indicated. This term generally refers to a range of numbers that one of ordinary skill in the art would consider as a reasonable amount of deviation to the recited numeric values (i.e., having the equivalent function or result). For example, this term can be construed as including a deviation of +10 percent of the given numeric value provided such a deviation does not alter the end function or result of the value. Therefore, a value of about 1% can be construed to be a range from 0.9% to 1.1%. Furthermore, a range may be construed to include the start and the end of the range. For example, a range of 10% to 20% (i.e., range of 10%-20%) can includes 10% and also includes 20%, and includes percentages in between 10% and 20%, unless explicitly stated otherwise herein.

It is understood that when combinations, subsets, groups, etc. of elements are disclosed (e.g., combinations of components in a composition, or combinations of steps in a method), that while specific reference of each of the various individual and collective combinations and permutations of these elements may not be explicitly disclosed, each is specifically contemplated and described herein.

Ranges can be expressed herein as from “about” one particular value, and/or to “about” another particular value. By “about” is meant within 5% of the value, e.g., within 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

Administration” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. “Concurrent administration”, “administration in combination”, “simultaneous administration” or “administered simultaneously” as used herein, means that the compounds are administered at the same point in time or essentially immediately following one another. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g. greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration but are undetectable or detectable at negligible amounts in distal parts of the subject's body. Administration includes self-administration and the administration by another.

As used here, the terms “beneficial agent” and “active agent” are used interchangeably herein to refer to a chemical compound or composition that has a beneficial biological effect. Beneficial biological effects include both therapeutic effects, i.e., treatment of a disorder or other undesirable physiological condition, and prophylactic effects, i.e., prevention of a disorder or other undesirable physiological condition. The terms also encompass pharmaceutically acceptable, pharmacologically active derivatives of beneficial agents specifically mentioned herein, including, but not limited to, salts, esters, amides, prodrugs, active metabolites, isomers, fragments, analogs, and the like. When the terms “beneficial agent” or “active agent” are used, then, or when a particular agent is specifically identified, it is to be understood that the term includes the agent per se as well as pharmaceutically acceptable, pharmacologically active salts, esters, amides, prodrugs, conjugates, active metabolites, isomers, fragments, analogs, etc.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

“Inactivate”, “inactivating” and “inactivation” means to decrease or eliminate an activity, response, condition, disease, or other biological parameter due to a chemical (covalent bond formation) between the ligand and a its biological target.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard of a control.

As used herein, the terms “treating” or “treatment” of a subject includes the administration of a drug to a subject with the purpose of preventing, curing, healing, alleviating, relieving, altering, remedying, ameliorating, improving, stabilizing or affecting a disease or disorder, or a symptom of a disease or disorder. The terms “treating” and “treatment” can also refer to reduction in severity and/or frequency of symptoms, elimination of symptoms and/or underlying cause, prevention of the occurrence of symptoms and/or their underlying cause, and improvement or remediation of damage. In particular, the term “treatment” includes the alleviation, in part or in whole, of the symptoms of coronavirus infection (e.g., sore throat, blocked and/or runny nose, cough and/or elevated temperature associated with a common cold). Such treatment may include eradication, or slowing of population growth, of a microbial agent associated with inflammation.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed. For example, the terms “prevent” or “suppress” can refer to a treatment that forestalls or slows the onset of a disease or condition or reduced the severity of the disease or condition. Thus, if a treatment can treat a disease in a subject having symptoms of the disease, it can also prevent or suppress that disease in a subject who has yet to suffer some or all of the symptoms. As used herein, the term “preventing” a disorder or unwanted physiological event in a subject refers specifically to the prevention of the occurrence of symptoms and/or their underlying cause, wherein the subject may or may not exhibit heightened susceptibility to the disorder or event. In particular embodiments, “prevention” includes reduction in risk of coronavirus infection in patients. However, it will be appreciated that such prevention may not be absolute, i.e., it may not prevent all such patients developing a disease, or may only partially prevent a disease in a single individual. As such, the terms “prevention” and “prophylaxis” may be used interchangeably.

By the term “effective amount” of a therapeutic agent is meant a nontoxic but sufficient amount of a beneficial agent to provide the desired effect. The amount of beneficial agent that is “effective” will vary from subject to subject, depending on the age and general condition of the subject, the particular beneficial agent or agents, and the like. Thus, it is not always possible to specify an exact “effective amount”. However, an appropriate “effective’ amount in any subject case may be determined by one of ordinary skill in the art using routine experimentation. Also, as used herein, and unless specifically stated otherwise, an “effective amount” of a beneficial can also refer to an amount covering both therapeutically effective amounts and prophylactically effective amounts.

An “effective amount” of a drug necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

As used herein, a “therapeutically effective amount” of a therapeutic agent refers to an amount that is effective to achieve a desired therapeutic result, and a “prophylactically effective amount” of a therapeutic agent refers to an amount that is effective to prevent an unwanted physiological condition. Therapeutically effective and prophylactically effective amounts of a given therapeutic agent will typically vary with respect to factors such as the type and severity of the disorder or disease being treated and the age, gender, and weight of the subject. The term “therapeutically effective amount” can also refer to an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the drug and/or drug formulation to be administered (e.g., the potency of the therapeutic agent (drug), the concentration of drug in the formulation, and the like), and a variety of other factors that are appreciated by those of ordinary skill in the art.

As used herein, the term “pharmaceutically acceptable” component can refer to a component that is not biologically or otherwise undesirable, i.e., the component may be incorporated into a pharmaceutical formulation of the invention and administered to a subject as described herein without causing any significant undesirable biological effects or interacting in a deleterious manner with any of the other components of the formulation in which it is contained. When the term “pharmaceutically acceptable” is used to refer to an excipient, it is generally implied that the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

As used herein, “pharmaceutically acceptable salt” is a derivative of the disclosed compound in which the parent compound is modified by making inorganic and organic, non-toxic, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are typical, where practicable. Salts of the present compounds further include solvates of the compounds and of the compound salts.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like, or using a different acid that produces the same counterion. Lists of additional suitable salts may be found, e.g., in Remington's Pharmaceutical Sciences, 17th ed., Mack Publishing Company, Easton, Pa., p. 1418 (1985).

Also, as used herein, the term “pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

A “control” is an alternative subject or sample used in an experiment for comparison purposes. A control can be “positive” or “negative.”

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human. Thus, the subject can be a human or veterinary patient. The term “patient” refers to a subject under the treatment of a clinician, e.g., physician. Administration of the therapeutic agents can be carried out at dosages and for periods of time effective for treatment of a subject. In some embodiments, the subject is a human.

### Abbreviations

FDA Food and Drug Administration
HBOC Hemoglobin-based oxygen carrier
bHb Bovine hemoglobin
MOF Metal organic framework
ZIF-8 Zeolitic imidazolate framework-8 (C₈H₁₂N₄Zn)
ZIF-8P-Hb Encapsulated hemoglobin using ZIF-8 precursors
PolybHb Polymerized bovine hemoglobin
DI Deionized water
DLS Dynamic light scattering
Hb Hemoglobin
HF Hollow fiber
hHb Human hemoglobin
Hp Haptoglobin
k_{O2.off} O₂ dissociation rate constant
MetHb Methemoglobin
MW Molecular weight
n Hill coefficient
NO Nitric oxide
P₅₀ Partial pressure of O₂ at which 50% of the hemoglobin is saturated with O₂
PB Phosphate buffer
PBS Phosphate buffered saline
pO₂ Partial pressure of O₂
PolyHb Polymerized hemoglobin
R-State Relaxed quaternary state
T-State Tense quaternary state
RBC Red blood cell
HPLC-SEC Size exclusion high performance liquid chromatography
TFF Tangential flow filtration
HbV Vesicle encapsulated hemoglobin

### Methods

Provided herein are method for producing a population of matrix-encapsulated protein particles. These methods can comprise: (a) combining a first framework precursor, a second framework precursor, and a protein to form a reactant mixture; (b) incubating the reactant mixture under conditions effective to form the population of matrix-encapsulated protein particles; and (c) separating the matrix-encapsulated protein from the reactant mixture using ultrafiltration. The resulting matrix-encapsulated protein particles can comprise a protein encapsulated in a porous framework formed by reaction of the first framework precursor and the second framework precursor.

In some embodiments, step (c) comprises filtering the reactant mixture comprising the matrix-encapsulated protein particles by ultrafiltration against a filtration membrane, thereby forming a retentate fraction comprising matrix-encapsulated protein particles having a molecular weight above a cutoff value and a permeate fraction comprising unencapsulated protein and other impurities having a molecular weight of less than the cutoff value.

The cutoff value can be between the molecular weight of the protein present in the reaction mixture and the average particle size of the matrix-encapsulated protein particles (i.e., so as to facilitate efficient separation of the matrix-encapsulated protein particles from unencapsulated protein and other impurities remaining in the reactant mixture). In some embodiments, the cutoff value is from 50 kDa to 1000 kDa (e.g., from 150 kDa to 750 kDa, from 250 to 750 kDa, or from 400 kDa to 600 kDa). In certain embodiments, the filtration membrane can be rated for retaining solutes having a molecular weight of greater than 50 kDa, such as greater than 100 kDa, greater than 150 kDa, greater than 250 kDa, greater than 300 kDa, or greater than 500 kDa.

In certain embodiments, the ultrafiltration can comprise tangential flow filtration or cross-flow filtration.

The porous framework can comprise, for example, a metal-organic framework (MOF), metal-inorganic framework (MIF), and/or covalent-organic framework (COF). In certain embodiments, the porous framework can comprise a MOF.

In some embodiments, the first framework precursor can comprise a metal salt and the second framework precursor can comprise a ligand. For example, in some examples, the first framework precursor can comprise a Fe salt, a Co salt, a Cu salt, a Zn salt, or a combination thereof. In some examples, the second precursor can comprise a ligand selected from the group consisting of imidazoles and derivatives such as 2-methylimidazole, 2-ethylimidazole, 4-azabenzimidazole, benzimidazole, nitroimidazole, 2-chloroimidazole, and the like; carboxylic acids and derivatives such as 1,4-benzenedicarboxylic acid, 1,3,5-benzene tricarboxylic acid, imidazole carboxaldehyde, 2-aminobenzimidazolate, the like, or any combination thereof.

In some embodiments, the first framework precursor and the second framework precursor can be present in the reactant mixture at a molar ratio of from 1:1 to 75:1, such as from 1:1 to 60:1, from 1:1 to 30:1, or from 15:1 to 30:1.

In certain embodiments, the porous framework can comprise a zeolitic imidazolate framework (ZIF). For example, the porous framework can comprise a zeolitic imidazolate framework such as ZIF-2, ZIF-3, ZIF-4, ZIF-8, ZIF-9, ZIF-10, ZIF-11, ZIF-12, ZIF-14, ZIF-20, ZIF-21, ZIF-23, ZIF-60, ZIF-61, ZIF-62, ZIF-64, ZIF-65, ZIF-67, ZIF-68, ZIF-69, ZIF-70, ZIF-71, ZIF-72, ZIF-73, ZIF-74, ZIF-75, ZIF-76, ZIF-77, ZIF-90, derivatives thereof, and combinations thereof.

The protein can comprise any suitable protein. For example, the protein can comprise conalbumin, albumin, hemoglobin, haptoglobin, hemopexin, transferrin, methemoglobin, ovalbumin, α-chymotrypsinogen A, α-chymotrypsin, trypsin, trypsinogen, β-lactoglobulin, myoglobin, α-lactalbumin, lysozyme, ribonuclease A, or cytochrome c, a recombinant version thereof, or a combination thereof. In some embodiments, the protein can be surface conjugated.

In some embodiments, the protein can comprise a globular protein. In certain embodiments, the protein can comprise hemoglobin. The hemoglobin can be from a mammalian, invertebrate, or recombinant source. For example, the hemoglobin can comprise human hemoglobin, bovine hemoglobin, or porcine hemoglobin. In some embodiments, the hemoglobin can comprise a polymerized hemoglobin. The polymerized hemoglobin can be in the tense or relaxed quaternary state, or is in between these two quaternary states.

In some embodiments, the reactant mixture can further comprise an etching agent, a chelating agent, or a combination thereof. For example, the etching agent can comprise hydrofluoric acid (HF), ammonium fluoride (NH₄F), the acid salt of ammonium fluoride

(NH₄HF₂), sodium hydroxide (NaOH), nitric acid (HNO₃), hydrochloric acid (HCl), hydroiodic acid (HI), hydrobromic acid (HBr), boron trifluoride (BF₃), sulfuric acid (H₂SO₄), acetic acid (CH₃COOH), formic acid (HCOOH), phosphoric acid (H₃PO₄), or any combination thereof. The chelating agent can comprise, for example, ethylenediaminetetraacetic acid (EDTA) or a derivative thereof.

The resulting population of matrix-encapsulated protein particles can have any suitable size. In some cases, the population of matrix-encapsulated protein particles can comprise microparticles. In other embodiments, the population of matrix-encapsulated protein particles can comprise nanoparticles.

In certain embodiments, the population of matrix-encapsulated protein particles can have an average particle size, as determined by electron microscopy, of less than 200 nm, such as less than 180 nm, less than 160 nm, less than 140 nm, less than 120 nm, less than 100 nm, or less than 80 nm.

In other embodiments, the population of matrix-encapsulated particles can have a larger average particle size, such as an average particle size of at least 500 nm, at least 750 nm, at least 1 micron, at least 1.5 microns, at least 2 microns, at least 2.5 microns, at least 5 microns, at least 10 microns, at least 20 microns, at least 30 microns, at least 40 microns, at least 50 microns, or at least 100 microns. These larger particles can be suitable for extracorporeal applications. By way of example, in some examples, these larger particles can include methemoglobin. Such particles can be used to scavenge, for example cyanide, hydrogen sulfide and/or azide, extracorporeally.

In some embodiments, the population of matrix-encapsulated protein particles can have a PDI of less than 0.100, such as less than 0.095, such as less than 0.090, less than 0.085, less than 0.080, less than 0.075, or less than 0.070.

In some embodiments, the population of matrix-encapsulated protein particles can have a zeta potential of less than −5 mV, such as of less than −6 mV, less than −7 mV, less than −8 mV, less than −9 mV, less than −10 mV, less than −11 mV, less than −12 mV, less than −13 mV, less than −14 mV, or less than −15 m V.

In some embodiments, the protein can retain its biological activity following encapsulation. For example, in some embodiments, at least 90% of the of the protein in the population of matrix-encapsulated protein particles retain their biological activity. For example, in the case of methods related to the encapsulation of hemoglobin, in some embodiments, at least 90% of the of the protein in the population of matrix-encapsulated protein particles comprises hemoglobin (and less than 10% of the protein comprises methemoglobin).

In some embodiments, the methods can encapsulate a protein with relatively high encapsulation efficiency. For example, in some embodiments, the method can encapsulate a protein with an encapsulation efficiency, measured by the fraction of the mass of the protein in the resulting matrix-encapsulated protein over the total mass of protein initially charged in the reactant mixture, of at least 80%, such as at least 82%, at least 84%, at least 86%, at least 88%, at least 90%, at least 92%, at least 94%, or at least 96%.

In some embodiments, the protein present in the population of matrix-encapsulated protein particles can be modified (e.g., oxidized, reduced, or covalently modified) before and/or after encapsulation. For example, provided are methods for producing a population of matrix-encapsulated protein particles, wherein the protein comprises methemoglobin or polymerized methemoglobin. In some embodiments, these methods can comprise combining a first framework precursor, a second framework precursor, and hemoglobin or polymerized hemoglobin to form a reactant mixture; incubating the reactant mixture under conditions effective to form the population of matrix-encapsulated protein particles; contacting the population of matrix-encapsulated protein particles with an oxidizing agent under conditions effective to convert the hemoglobin or polymerized hemoglobin to methemoglobin or polymerized methemoglobin; and separating the matrix-encapsulated protein from the reactant mixture using ultrafiltration; wherein the matrix-encapsulated protein particles comprise a protein encapsulated in a porous framework formed by reaction of the first framework precursor and the second framework precursor. In these embodiments, the step of contacting the matrix-encapsulated protein particles with an oxidizing agent can be performed before and/or after the step of separating the matrix-encapsulated protein from the reactant mixture. In other embodiments, these methods can comprise contacting hemoglobin or polymerized hemoglobin with an oxidizing agent under conditions effective to convert the hemoglobin or the polymerized hemoglobin to methemoglobin or polymerized methemoglobin; combining a first framework precursor, a second framework precursor, and the methemoglobin or the polymerized methemoglobin to form a reactant mixture; incubating the reactant mixture under conditions effective to form the population of matrix-encapsulated protein particles; and separating the matrix-encapsulated protein from the reactant mixture using ultrafiltration; wherein the matrix-encapsulated protein particles comprise methemoglobin or polymerized methemoglobin encapsulated in a porous framework formed by reaction of the first framework precursor and the second framework precursor. In some of the above-referenced embodiments, the oxidizing agent can comprise a weak oxidizing agent, such as potassium nitrite or sodium nitrite. In some of the above-referenced embodiments, the polymerized methemoglobin can be in the tense or relaxed quaternary state, or is in between these two quaternary states.

Also provided are pharmaceutical compositions comprising a population of matrix-encapsulated hemoglobin or polymerized hemoglobin particles prepared using the methods described herein. The compositions can be administered to a subject in need thereof, for example, to treat hypoxia (e.g., hypoxia is at least partially caused by traumatic brain injury or hemorrhagic shock).

Also provided are pharmaceutical compositions comprising a population of matrix-encapsulated methemoglobin or polymerized methemoglobin particles prepared using the methods described herein. These compositions can be administered to a subject in need thereof, for example, to treat cyanide, hydrogen sulfide, and/or azide poisoning. A population of matrix-encapsulated methemoglobin or polymerized methemoglobin particles can also be employed extracorporeally to treat cyanide, hydrogen sulfide, and/or azide poisoning.

Reference will now be made in detail to specific aspects of the disclosed materials, compounds, compositions, articles, and methods, examples of which are illustrated in the accompanying Examples and Figures.

### EXAMPLES

### Example 1: ZIF-8 Metal Organic Framework Encapsulated Hemoglobin

### Summary

Hemoglobin (Hb)-based oxygen carriers (HBOCs) are being developed as artificial red blood cell (RBC) substitutes for use in transfusion medicine. Unfortunately, prior generations of HBOCs were not able to successfully minimize key side effects including vasoconstriction, systemic hypertension and oxidative tissue injury, which is primarily due to the extravasation of cell-free Hb from the vascular space into the tissue space. Therefore, to potentially reduce these side effects, we successfully encapsulated Hb within a zeolitic imidazolate framework (ZIF-8) to form ZIF-8-Hb nanoparticles (ZIF-8P-Hb). Both ZIF-8 and ZIF-8P-Hb nanoparticles were synthesized at a relatively high molar ratio of 2-methylimidazole:zinc, which resulted in a monodisperse nanoparticle size distribution. In addition, the flow conditions for tangential flow filtration facilitated purification of the nanoparticles did not exert a strong effect on the nanoparticle size distribution. ZIF-8P-Hb nanoparticles exhibited high stability, ultrahigh Hb encapsulation efficiency and a monodisperse size distribution. Additionally, ZIF-8P-Hb nanoparticles exhibited a zeta potential of −11.2±0.9 mV, demonstrating its potentially enhanced biocompatibility in comparison to bare ZIF-8 nanoparticles (40.7±2.0 mV). More significantly, ZIF-8P-Hb nanoparticles exhibited significantly enhanced hydrothermal stability with negligible release of cell-free Hb. Furthermore, ZIF-8P-Hb displayed a significantly lower haptoglobin binding rate constant compared to cell-free Hb, indicating its potentially slower in vivo clearance in comparison to cell-free Hb. Moreover, we observed a relatively low level of hemolysis when ZIF-8P-Hb nanoparticles were incubated with RBCs (<5%), which demonstrate a suitable safety profile. To further optimize the ZIF-8P-Hb nanoparticle synthesis protocol, various procedural parameters were systematically investigated to evaluate their impact on the size distribution of ZIF-8 and ZIF-8P-Hb nanoparticles. Taken together, this work provides a comprehensive approach for synthesizing a monodisperse HBOC as a potential artificial RBC substitute.

### Introduction

Metal organic frameworks (MOFs) are crystalline materials synthesized by the coordination of metal ions and organic linkers. Zeolitic imidazolate frameworks (ZIFs) are a subclass of MOF, which exhibits outstanding solvothermal stability due to their unique structure compared to other MOFs. ZIF-8, the prototypical ZIF, preserves its crystallinity and zeolite-like porosity when exposed to boiling water, organic solvents, and several biological buffers. Very recently, ZIF-8 has emerged as a promising material in biological sensing, and pharmaceutical applications.

Recently, ZIF-8 encapsulated Hb (ZIF-8@Hb) nanoparticles have been prepared, which improved survival in a murine hemorrhagic shock model compared to that of transfused cell-free Hb. Despite relatively high Hb encapsulation efficiency, the absolute amount of Hb loaded into ZIF-8 nanoparticles was quite low likely due to the interference of Hb on ZIF-8 nanoparticle nucleation. Additionally, the final ZIF-8@Hb nanoparticle suspension exhibited a dark green color, which qualitatively indicated excessive formation of methemoglobin (metHb). MetHb is the oxidized form of ferrous Hb, which cannot bind O₂.

In this Example, bovine Hb (bHb) was successfully encapsulated using ZIF-8 precursors into ZIF-8P-Hb nanoparticles and exhibited low oxygen affinity. Bovine Hb encapsulation was performed in the presence of a 30:1 molar ratio of 2-methylimidazole (Hmim) to zinc to reduce ZIF-8P-Hb nanoparticle polydispersity and size by facilitating the nanoparticle nucleation process. ZIF-8P-Hb nanoparticles were then purified via tangential flow filtration (TFF) to remove unreacted ligands and residual cell-free Hb. TFF was found to have more flexibility and scalability compared to conventional centrifugation method since centrifuges have a finite limit on the size of the containers, hindering the overall feasibility of large scale manufacturing via centrifugation. ZIF-8P-Hb nanoparticles were able to maintain their oxygen binding/release capabilities. Moreover, protection of encapsulated Hb against recognition by haptoglobin was found to be substantial given that the haptoglobin binding rate constant was significantly lower for ZIF-8P-Hb nanoparticles compared to that of cell-free bHb. Furthermore, the significantly reduced zeta potential of ZIF-8P-Hb nanoparticles compared to bare ZIF-8 nanoparticles suggests potential biocompatibility. Finally, the low level of hemolysis (<5%) induced by ZIF-8P-Hb nanoparticles demonstrate suitable hemocompatibility for its potential use as an RBC substitute in treating hemorrhagic shock. Further optimization was performed by evaluating the effect of multiple procedural parameters on the size distribution and polydispersity of ZIF-8 and ZIF-8P-Hb nanoparticles.

### Methods

### Bovine Hb Purification

Bovine Hb (bHb) was purified via tangential flow filtration (TFF) as described previously in the literature. Briefly, Hb was purified via a two-stage TFF system with hollow fiber cartridges with MWCOs of 500 and 50 kDa (Repligen Corporation, Rancho Dominguez, CA). The purified bHb was concentrated to >200 mg/mL and stored at −80° C. for future use.

### Synthesis and Purification of ZIF-8-Hb Nanoparticles

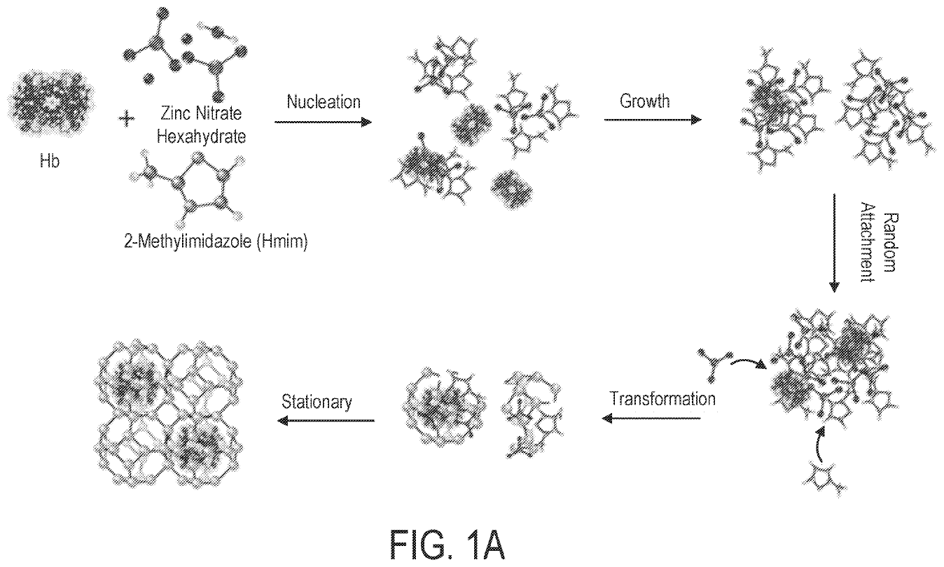

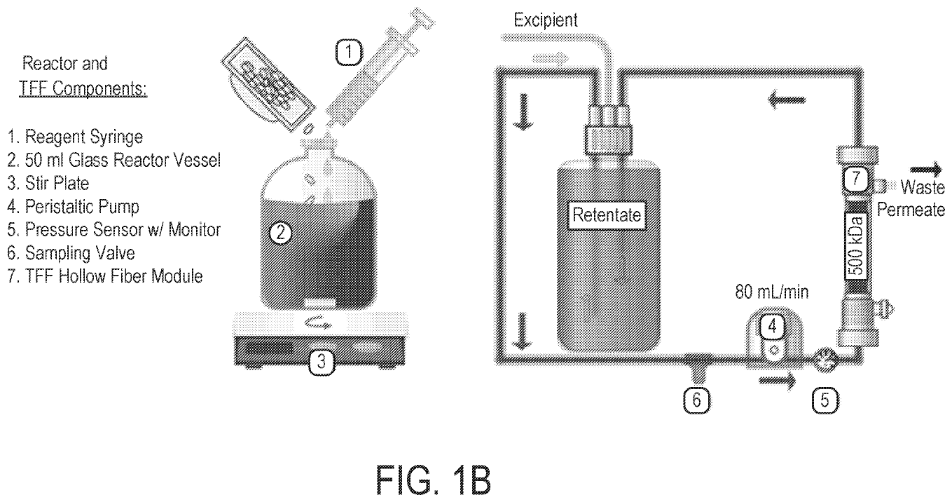

FIGS. 1A-1B illustrate the synthesis schematic of nanoparticle encapsulated bHb using ZIF-8 precursors (ZIF-8P-Hb). Initially, 50 mL of deionized (DI) water was used to fully dissolve 100 mg of Zn(NO₃)₂·6H₂O, chased by a 1 mL bolus addition of bHb solution (250 mg/mL) with continuous stirring (500 rpm) for 5 min. 827.9 mg of Hmim powder was then added to the above mixture. The reaction proceeded for 1 h at 25° C. followed by overnight stabilization at 4° C. Then, the formed ZIF-8P-Hb nanoparticles were purified by washing with TFF for 6˜10 diacycles using a hollow fiber cartridge with 500 kDa pore size first with DI water and buffered exchanged into phosphate buffered saline (PBS, 0.1 M) for further use. Bare ZIF-8 nanoparticles were synthesized following a similar protocol without the addition of bHb and were suspended in DI water.

### Synthesis Parameter Optimization

To optimize the protocol for synthesizing ZIF-8 and ZIF-8P-Hb nanoparticles, the effect of various synthesis parameters on the biophysical properties of ZIF-8/ZIF-8P-Hb nanoparticles were evaluated. More specifically, the initial concentration of bHb, the concentration of EDTA, the flow rate of the peristaltic pump during the diafiltration process, the concentration of zinc nitrate, the molar ratio of Hmim:Zn, and different addition methods for introduction of Hmim and zinc nitrate hexahydrate into the reaction vessel were varied in this study

### Hydrodynamic Diameter

The hydrodynamic diameter of bHb, ZIF-8 and ZIF-8P-Hb nanoparticles were measured using a BI-200SM goniometer (Brookhaven Instruments Corp., Holtsville, NY) at an angle of 90° and wavelength of 637 nm. Protein samples were diluted to ˜0.5-1 mg/mL concentration in DI water. The hydrodynamic diameter was calculated via the instrument software.

### Morphology and Crystalline Structure

The morphology of ZIF-8 and ZIF-8P-Hb nanoparticles was studied with a Thermo Glacios cryo-electron microscopy (Thermo Fisher Scientific). Protein samples were diluted to ˜0.5-1 mg/mL concentration in DI water. XRD patterns were recorded on a Bruker D8 Advance diffractometer (AXS, Bruker, Germany) with Cu target from 5° C. to 55° C.

### Oxygen (O₂) Equilibrium Curves

O₂ equilibrium curves (OECs) for bHb and ZIF-8P-Hb nanoparticles were measured using a Hemox Analyzer (TCS Scientific Corp., New Hope, PA) at 37.0±0.1° C. in phosphate buffered saline (PBS, 0.1 M, pH 7.4). To quantify the oxygen binding affinity (P₅₀) and cooperativity coefficient (n), the OEC was fit to the Hill equation to regress the parameters as described in the literature.

### Rapid Deoxygenation Kinetics

Initially, bHb and ZIF-8P-Hb nanoparticles were diluted to 12.5 μM (heme basis) in PBS (0.1 M, pH 7.4). Deoxygenated buffer was prepared by adding 1.5 mg/mL of sodium dithionite to PBS bubbled under N₂ for 30 minutes. Deoxygenated buffer and either oxygenated bHb orZIF-8P-Hb nanoparticles were mixed rapidly in a microvolume stopped-flow spectrophotometer (Applied Photophysics Ltd., Surrey, United Kingdom) and the absorbance was monitored at 437.5 nm. An exponential decay function was fit to the data and the rate constant for O₂ dissociation (k_{off.O2}) was regressed for each sample.

### Haptoglobin Binding Kinetics

The kinetics of haptoglobin (Hp) binding to bHb or ZIF-8P-Hb nanoparticles was measured in PBS (0.1 M, pH 7.4) as described in the literature. The reaction between Hp and bHb or ZIF-8P-Hb nanoparticles was monitored by stopped flow fluorescence spectrometry (λ_{excitation}=285 nm, λₑₘᵢₛₛᵢₒₙ=310 nm) as previously described in the literature. The pseudo first order Hp binding rate constant was calculated by fitting the fluorescence intensity to a mono-exponential equation. The pseudo first-order rate constants as a function of [Hb] was then used to determine the bimolecular rate constant via linear regression.

Hb Release from ZIF-8P-Hb Nanoparticles

Hb release from ZIF-8P-Hb nanoparticles was studied as a function of the bHb release rate as described in the literature. Briefly, bHb release from ZIF-8P-Hb nanoparticles was measured over 14 days at 4° C. in PBS (0.1 M, pH 7.4). The permeate was collected by filtering the stored nanoparticle solution through a 500 kDa TFF membrane. The concentration of bHb from the resulting permeate was measured using UV-visible spectroscopy.

### Hemocompatibility of ZIF-8P-Hb Nanoparticles

Expired human RBCs and plasma units were generously donated by Transfusion Services, Wexner Medical Center, The Ohio State University (Columbus, OH). The hemolysis assay was performed as described in the literature. Briefly, 1 mL of human RBCs was mixed with 1 mL of ZIF-8P-Hb nanoparticles suspended in 0.9% saline solution. The mixtures were incubated at 37° C. in a water bath incubator for 30 minutes and then centrifuged to collect the supernatant. The bHb concentration of the supernatant was measured with UV-visible spectroscopy. Hemolysis was determined by the ratio of supernatant concentration of bHb in ZIF-8P-Hb nanoparticle/RBC mixture to the total Hb concentration derived from the RBCs.

### Total bHb and Methemoglobin (MetHb) Levels

Total bHb and metHb concentrations were determined using the cyanmethemoglobin method. To accurately measure the bHb concentration and reduce the effect of nanoparticle scattering, the cyanmethemoglobin method was slightly modified by adding EDTA (0.5M) to fully dissolve the ZIF-8P-Hb nanoparticle crystalline structure prior to the addition of cyanide. Spectrophotometric absorbance measurements were obtained using a HP 8452A diode array spectrophotometer (Olis, Bogart, GA).

### bHb Encapsulation Efficiency and Loading

The encapsulation efficiency (EE %) and loading of bHb inside ZIF-8P-Hb nanoparticles was calculated based on the total mass fraction of encapsulated bHb present in the retentate solution as shown in Eqs 1 and 2, respectively, where Hbᵢₙᵢₜ corresponds to the mass of bHb in the permeate solution at the end of the TFF washing process and Hbᵢₙᵢₜ corresponds to the initial mass of bHb.

$$EE ( \% ) = ( \frac{H {b}_{init} - {Hb}_{per}}{H {b}_{init}} ) \times 100 \% \qquad ( Eq 1 )$$

$$Hb loading ( HL ) = ( \frac{{Hb}_{init} - {Hb}_{per}}{total mass of ZIF - 8 P - Hb} ) \qquad ( Eq 2 )$$

### Statistical Analysis

In this study, all statistical analysis was performed using a t-test, and a p value of <0.05 was considered significant.

### Results and Discussion

Table 1 lists the biophysical properties of ZIF-8 and ZIF-8P-Hb nanoparticles in comparison to other types of HBOCs. The effect of bHb encapsulation using ZIF-8 precursors on the biophysical properties of ZIF-8P-Hb nanoparticles was studied by comparing the hydrodynamic diameter, zeta potential, oxygen equilibria, oxygen offloading rate constant, and Hp binding rate constant to native bHb and other HBOCs from the literature.

TABLE 1
Biophysical properties of bHb, ZIF-8 and ZIF-8P-Hb nanoparticles, and other types of HBOCs described in the
literature including R-state PolybHb 30:1 (R30), T-state PolybHb 35:1 (T35), and vesicle encapsulated Hb
(HbV). R30 and T35 represent typical polymerized Hbs synthesized via glutaraldehyde cross-linking under
fully oxygenated (relaxed quaternary state [R]) and deoxygenated (tense quaternary state [T]) conditions
at glutaraldehyde:bHb molar ratios of 30:1 and 35:1. HbV represent a phospholipid bilayer membrane (liposome)
encapsulating an aqueous core of concentrated Hb molecules. The biophysical properties of RBCs was also
included in the table since it is the natural oxygen carrier in most organisms with a circulatory system.

| Parameter | bHb | ZIF-8 | ZIF-8P-Hb | R30 | T35 | HbV | RBCs |
|---|---|---|---|---|---|---|---|
| P₅₀ (mm Hg) | 26.3 ± 0.5 | — | 11.8 ± 0.6 | 1.2 ± 0.4 | 38.9 ± 2.5 | 32 | 25.6 |
| Cooperativity (n) | 2.50 ± 0.10 | — | 1.99 ± 0.08 | 1.0 ± 0.2 | 1.0 ± 0.1 | 2.5 | 2.8 |
| MW (kDa) | 65 | — | — | 1,131 ± 97 | 1,194 ± 106 | — | — |
| D_{eff} (nm) | 6.9 ± 0.3 | 90.3 ± 11.8 | 106.0 ± 9.7 | 62.4 ± 12.5 | 64.1 ± 9.3 | 251 ± 87 | 7-8 × 10³ |
| PDI | 0.1 | 0.065 ± 0.006 | 0.085 ± 0.008 | 0.24 ± 0.02 | 0.19 ± 0.02 | — | — |
| Zeta potential (mV) | −21.2 ± 2.5 | 40.7 ± 2.0 | −11.2 ± 0.9 | −11.1 ± 0.6 | −22.5 ± 1.3 | −18 | −12.5 |
| k_{off, O2} (s⁻¹) | 45.86 ± 1.00 | — | 19.57 ± 1.27 | 15.12 ± 1.60 | 35.13 ± 9.90 | 9.4 | 5.1 |
| k_{Hb-Hp} (μM⁻¹s⁻¹) | 0.1491 | — | 0.0405 | 0.0076 | 0.0228 | — | — |
| MetHb level (%) | 1.0 ± 0.3 | — | 4.1 ± 0.4 | 3.7 ± 1.1 | 3.2 ± 1.2 | <10 | <1 |

### Crystalline Structure and Morphology

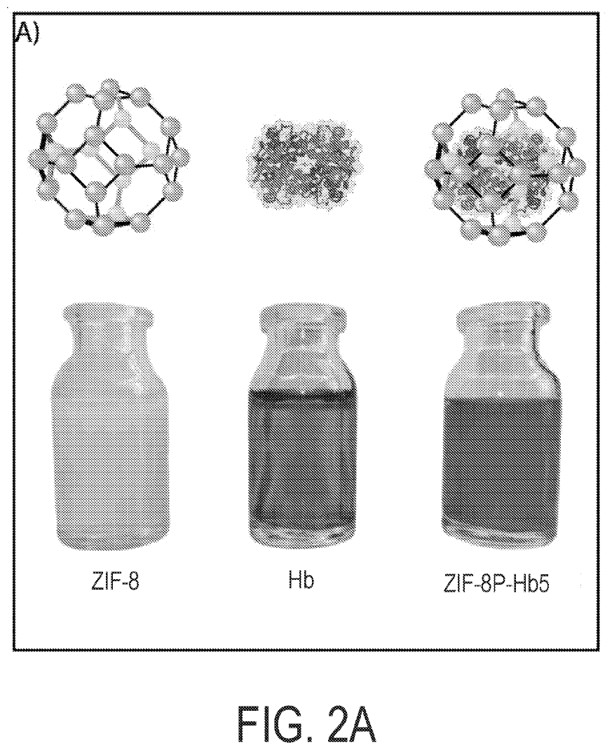

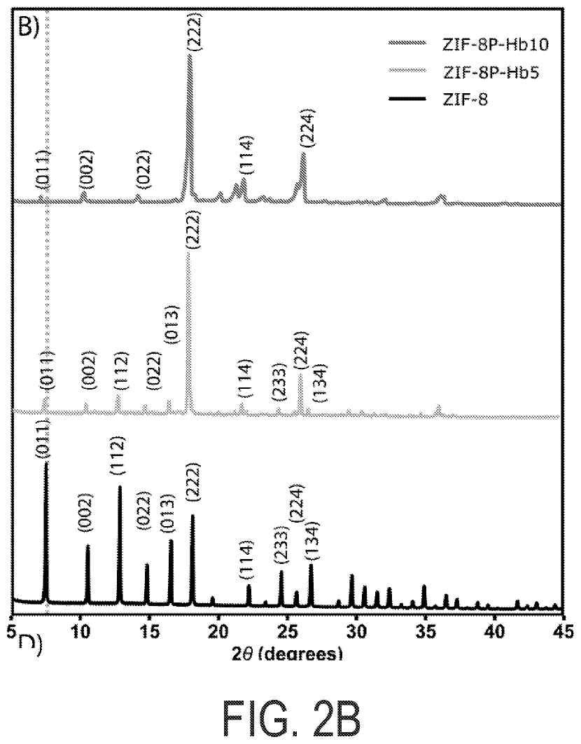

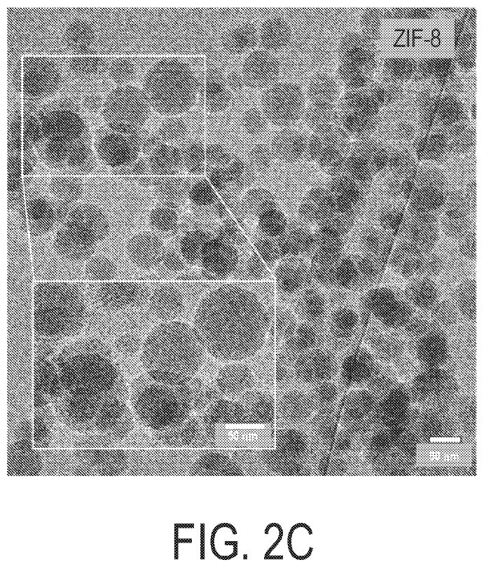

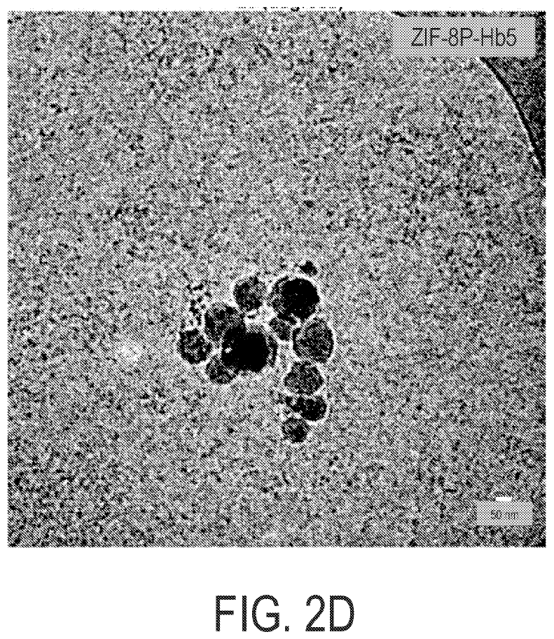

In FIG. 2A, the bare ZIF-8 nanoparticle solution exhibited a milky white color, whereas cell-free bHb exhibited a red color. After bHb encapsulation, the ZIF-8P-Hb 5 nanoparticle solution exhibited an opaque red color, resulting from nanoparticle formation. The characteristic diffraction peaks at 2θ=7.4°, 10.4°, 12.7°, 14.7°, 16.4°, 18.0°, 22.1°, 24.5°, and 26.7° for ZIF-8 nanoparticles were observed explicitly in FIG. 2B, which corresponded to (011), (002), (112), (022), (013), (222), (114), (233), and (134) planes, respectively. For ZIF-8P-Hb nanoparticles, the slightly left shifted diffraction peak positions at low 2θ angles compared to bare ZIF-8 nanoparticles, suggest a potential distortion of the ZIF-8 lattice, likely due to ZIF-8 framework expansion caused by the electrostatic interactions between the electrophile (Fe²⁺ from heme) and nucleophile (imidazole groups in ZIF-8 framework). For ZIF-8P-Hb nanoparticles, the PXRD patterns suggest that increasing the bHb content might introduce unknown phases in the ZIF-8 crystalline structure. The absence of several ZIF-8 nanoparticle diffraction peaks (e.g. 12.7°) in the ZIF-8P-Hb10 nanoparticles could be attributed to spacing differences between the lattice planes due to interactions with encapsulated bHb. To further confirm the morphology of ZIF-8 and ZIF-8P-Hb nanoparticles, cryo-electron microscopy (Cryo-EM) was used to image the nanoparticles as shown in FIGS. 2C and 2D. In FIG. 2C, ZIF-8 nanoparticles exhibited hexagonal nanocrystals which is consistent with previous observations in the literature. FIG. 2D reveals a slightly different shape for ZIF-8P-Hb nanoparticles, which could be a consequence of the crystalline structure distortion. It was observed that ZIF-8P-Hb nanoparticles possessed an average diameter of 112 nm (Cryo-EM), which was consistent with the value obtained via DLS (Table 1).

### Size and Zeta Potential

It was observed that ZIF-8 nanoparticles synthesized in this study possessed a hydrodynamic diameter of 90.3±11.8 nm with a relatively low polydispersity (PDI) of 0.065 #: 0.006 as shown in Table 1. After bHb encapsulation, ZIF-8P-Hb nanoparticles exhibited a hydrodynamic diameter of 106.0±9.68 nm with narrow size distribution (PDI=0.085±0.008). In comparison to ZIF-8@Hb nanoparticles (diameter ranging from 164.5-365.2 nm) synthesized in the literature, the size of ZIF-8P-Hb nanoparticles (diameter ˜100 nm) synthesized in this current study is more advantageous, since nanoparticles with larger particle sizes (>300 nm) are more prone to uptake by the reticuloendothelial system (RES) and being trapped in the hepatic sinusoids.

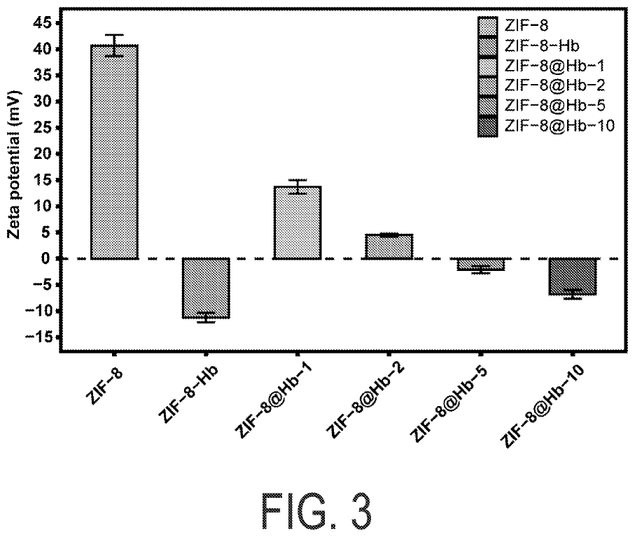

Surface charge changes were assessed by measuring the zeta potential before and after bHb encapsulation inside the ZIF-8 nanoparticle (Table 1). In FIG. 3, bHb encapsulation with ZIF-8 precursors reduced the zeta potential of ZIF-8-Hb nanoparticles from 40.7±2.04 mV (bare ZIF-8 nanoparticles) to −11.2±0.93 mV (ZIF-8P-Hb nanoparticles). The dramatic decrease of the zeta potential demonstrated successful encapsulation of bHb given that the zeta potential of cell-free bHb is −5.8±0.7 m V, which is consistent as previously reported in the literature. The positive charged ZIF-8 nanoparticles were neutralized by excessive amount of negatively charged Hb via encapsulation. In general nanoparticles with negative zeta potential possess relatively low cytotoxicity, likely due to the weak electrostatic interactions with cell membranes. Thus, it is reasonable to conclude that encapsulation of bHb using ZIF-8 precursors should increase the potential biocompatibility of ZIF-8-Hb nanoparticles just based on the favorable surface charge. ZIF-8P-Hb nanoparticles synthesized in this study also exhibited lower zeta potential than that of ZIF-8@Hb-1 (13.7±1.3), ZIF-8@Hb-2 (4.5±0.3), ZIF-8@Hb-5 (−2.1±0.7), and ZIF-8@Hb-10 (−6.8±0.8) nanoparticles reported in the literature. Such differences indicate higher bHb loading and potentially superior biocompatibility of ZIF-8P-Hb nanoparticles compared to previous formulations described in the literature.

### Procedural Meta-Data Analysis

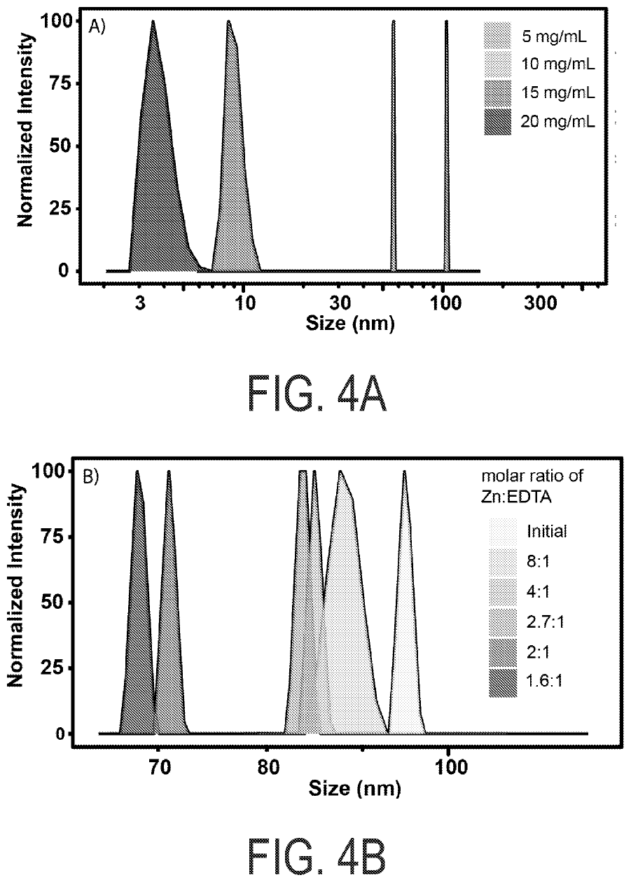

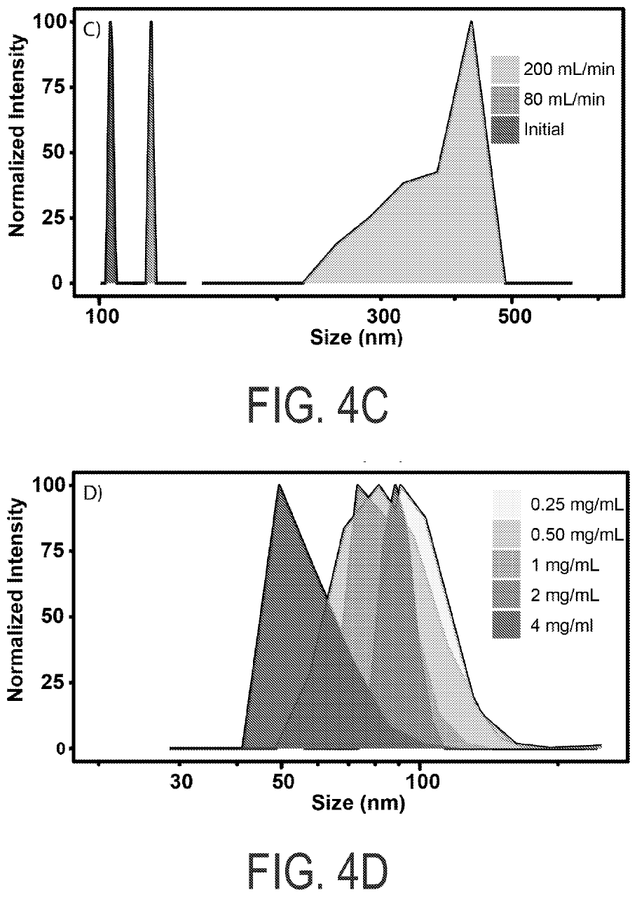

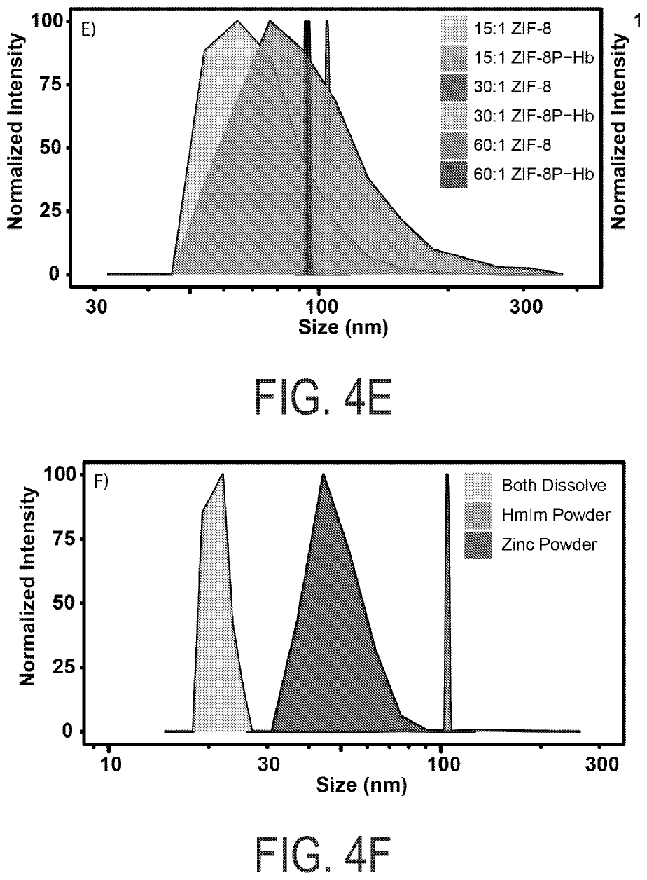

FIG. 4A shows the effect of the initial bHb concentration on the size of the resultant ZIF-8P-Hb nanoparticles. It was observed that increasing the concentration of bHb during synthesis yielded smaller nanoparticles, which could be attributed to interference of the bHb molecules on the ZIF-8P-Hb nanoparticle nucleation process. Thus, in this study, a bHb concentration of 5 mg/mL was chosen as the optimal Hb concentration for ZIF-8P-Hb nanoparticle synthesis, which resulted in particles with an average hydrodynamic diameter of 104 nm. FIG. 4B shows the etching effect of EDTA on the hydrodynamic diameter of ZIF-8. Previously, it was observed that bare ZIF-8 nanoparticles can be fully dissolved in EDTA (0.5 M). In this study, EDTA (80 mM, 7.40) was added during the nanoparticle synthesis process to evaluate its' etching effect on ZIF-8 nanoparticles. It was found that the size of ZIF-8 nanoparticles decreased when reducing the molar ratio of Zn:EDTA (from 8:1 to 1.6:1), demonstrating favorable particle diameter tunability using EDTA as an etching agent. FIG. 4C shows the effect of the TFF peristaltic pump flow rate during the TFF-facilitated nanoparticle washing process on the hydrodynamic diameter of the ZIF-8 nanoparticle. Running the TFF pump at 80 mL/min had a slightly impact on the size and polydispersity of the ZIF-8 nanoparticle (104.7 nm to 122.4 nm). Drastic aggregation was observed at a flowrate of 300 mL/min (372.4 nm). Higher pump flowrates seemed to cause more particle aggregation and heterogeneous formation of ZIF-8 nanoparticles likely due to increased flow-induced particle collisions which increase the collision efficiency between nanoparticles in solution.

FIG. 4D shows the effect of the initial concentration of zinc nitrate hexahydrate on the hydrodynamic diameter of ZIF-8 nanoparticles at a 30:1 molar ratio of Hmim:Zn. Decreasing the concentration of the zinc nitrate hexahydrate from 4 mg/mL to 2 mg/mL resulted in a significant change in the average size of ZIF-8 nanoparticles (from 58.2 nm to 89.9 nm). This behavior could be due to the fact that the high concentration of ZIF-8 precursor solution triggered a rapid coordination reaction, which yielded relatively small particles. In FIG. 4E, a broader size distribution of ZIF-8 nanoparticles was observed as the Hmim:Zn ratio increased from 15:1 to 60:1, which was observed during ZIF-8P-Hb synthesis. The relatively high Hmim:Zn molar ratio likely yielded ZIF-8 nanoparticles with low polydispersity, which was consistent with the literature. In comparison to 60:1 ZIF-8P-Hb nanoparticles (94.6 nm), 30:1 ZIF-8P-Hb nanoparticles possessed a slightly larger hydrodynamic diameter (110.2 nm), most likely due to the slower coordination reaction driven by the addition of less Hmim. Similar findings for ZIF-8 nanoparticles were reported in the literature. Therefore, the faster nucleation rate might impede the growth of ZIF-8 nanoparticles. In this study, we also tested the effect of different addition methods for introducing Hmim and zinc nitrate hexahydrate including adding zinc salts directly to the Hmim solution, adding Hmim directly to the zinc salt solution, and fully dissolving both and pouring the Hmim solution into the zinc salt solution (FIG. 4F). Both methods except the direct addition of Hmim yielded a broader particle size distribution, which shows the benefits of adding Hmim directly into the zinc salt solution.

### Oxygen Affinity and Offloading Kinetics

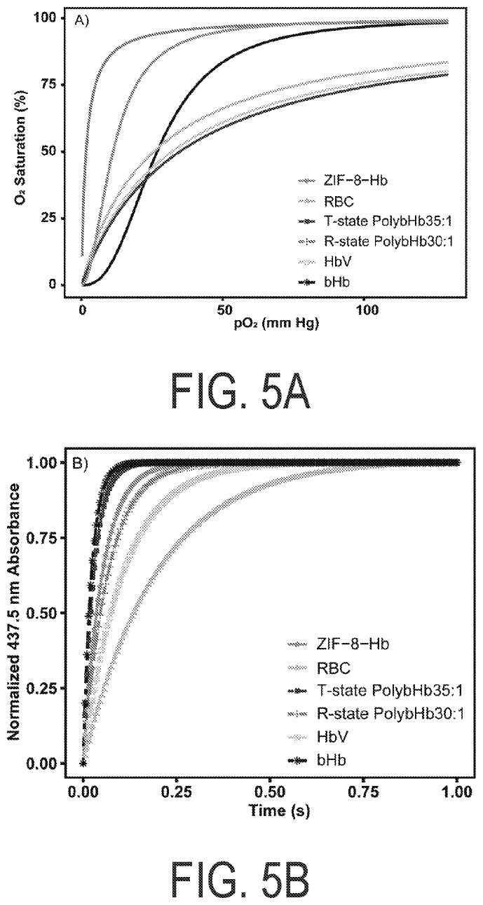

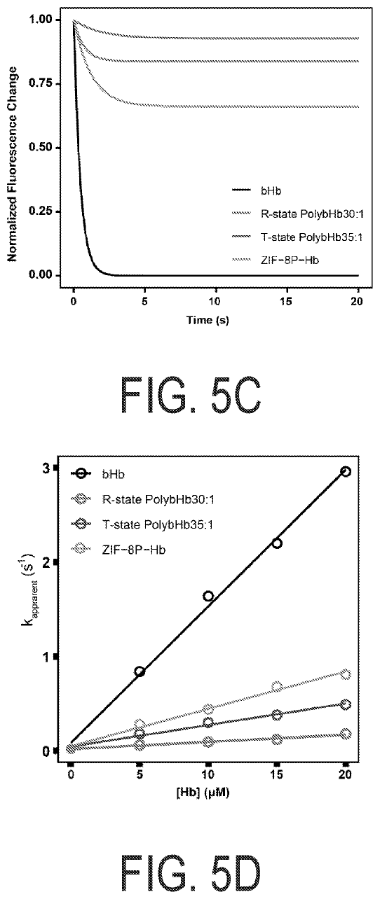

FIG. 5A shows the OBCs for bHb, ZIF-8P-Hb nanoparticles, and other types of HBOCs, from which the oxygen affinity (P₅₀) and Hill cooperativity coefficient (n) were calculated by fitting the OECs to the Hill equation. In FIG. 5A, ZIF-8P-Hb nanoparticles exhibited a left shifted OEC, indicating higher oxygen affinity in comparison to bHb (˜26.5 mm Hg), RBCs (˜25.6 mm Hg), and HbV (˜32 mm Hg). This could be a consequence of the hydrogen bonding between the oxygen atom in ferrous heme (Fe²⁺) and the amine group contained in Hmim. Given that both Hmim and the distal histidine (His E7) of bHb possess an imidazole ring with an amine group, Hmim might compete with His E7 to form hydrogen bonds with O₂. Hmim should likely form a stronger hydrogen bond, which increased the oxygen affinity as observed in FIG. 5A. Since the distal histidine (His E7) of bHb plays an important role in cooperative O₂ binding, competition between Hmim and His E7 in forming a hydrogen bond with O₂ might also affect the cooperativity. From Table 1, ZIF-8P-Hb nanoparticles exhibited lower cooperativity compared to free bHb, RBCs and HbV, which possessed a cooperativity of 2.50±0.10, ˜2.5 and ˜2.8 respectively. Interestingly, ZIF-8P-Hb nanoparticles exhibited a markedly higher cooperativity than that of both T-state PolybHb 35:1 (0.99±0.03) and R-state PolybHb 30:1 (1.2±0.4). This behavior could be due to the chemical cross-linking in the polymerized bHb superstructure which restricted conformational changes in the bHb tetramer.

The k_{off.O2} of ZIF-8P-Hb nanoparticles (19.57+1.27 s⁻¹) was significantly higher compared to HbV (9.4 s⁻¹) and RBCs (5.1 s⁻¹), which could be due to the porous structure of ZIF-8P-Hb nanoparticles which facilitated faster O₂ diffusion through the particle core (FIG. 5B). In comparison to bHb, ZIF-8P-Hb nanoparticles released Oz slower than native bHb. This could be due to Oz diffusion limitations through the pores and cavities of the ZIF-8P-Hb nanoparticles. It was also observed that the k_{off.O2} of ZIF-8P-Hb nanoparticles (19.57+1.27 s⁻¹) was lower than that of T-state PolybHb 35:1 (35.13±9.90 s⁻¹), but slightly higher than R-state PolybHb 30:1 (15.12±1.60 s⁻¹). This was expected given that ZIF-8P-Hb nanoparticles possessed a P₅₀ of 11.8±0.6 mm Hg, which is in between that of T-state PolybHb 35:1 (38.9+2.5 mm Hg) and R-state PolybHb 30:1 (1.2±0.4 mm Hg).

### Haptoglobin Binding Kinetics.

FIG. 5C displays the pseudo first order Hp binding kinetics of bHb, ZIF-8P-Hb nanoparticles, R-state PolybHb 30:1 and T-state PolybHb 35:1. In comparison to bHb, a significantly lower amount of Hp was quenched by ZIF-8P-Hb nanoparticles, which should potentially lead to slower in vivo clearance by CD163+ macrophages and monocytes. Figure SD shows a linear regression of the pseudo first order binding rate constants as a function of the bHb concentration. Specifically, a much lower k_{Hp-Hb} was observed for ZIF-8P-Hb nanoparticles (0.0405 μM⁻¹ s⁻¹) compared to cell-free bHb (0.1491 μM⁻¹ s⁻¹), indicating successful encapsulation of bHb which prevent direct exposure to Hp. In comparison to T-state PolybHb 35:1 (0.0228 μM⁻¹ s⁻¹) and R-state PolybHb 30:1 (0.0076 μM⁻¹ s⁻¹), ZIF-8P-Hb nanoparticles exhibited higher k_{Hb-Hp} (0.0405 μM⁻¹ s⁻¹). This behavior could be attributed to the cell-free bHb trapped on the nanoparticle surface.

### bHb Encapsulation Efficiency and bHb Leakage

In general, cell-free bHb not removed by diafiltration would be included in the quantity of particle entrapped Hb as shown in Eq 1. In this study, ultrahigh bHb encapsulation efficiency (EE %=88.2±3.5%) of ZIF-8P-Hb nanoparticles was achieved, indicating favorable oxygen carrying capacity. In comparison to the EE % of HbV prepared via both the conventional extrusion method (˜20%) and the kneading method (˜74.2%), the encapsulation of bHb using ZIF-8 precursors yielded a significantly higher EE % likely because the nucleation and encapsulation process were both rapid due to the high molar ratio of Hmim:Zn (30:1). ZIF-8P-Hb nanoparticles also exhibited higher EE % and Hb loading (˜0.81 mg Hb/1 mg ZIF-8P-Hb) than that of ZIF-8@Hb nanoparticles (EE %=82.1%, Hb loading=˜0.03-0.24 mg Hb/1 mg ZIF-8@Hb) as described in the literature. The Hb loading of ZIF-8@Hb nanoparticles was estimated by assuming 100% yield of the ZIF-8@Hb nanoparticles based on the mass of the zinc source.

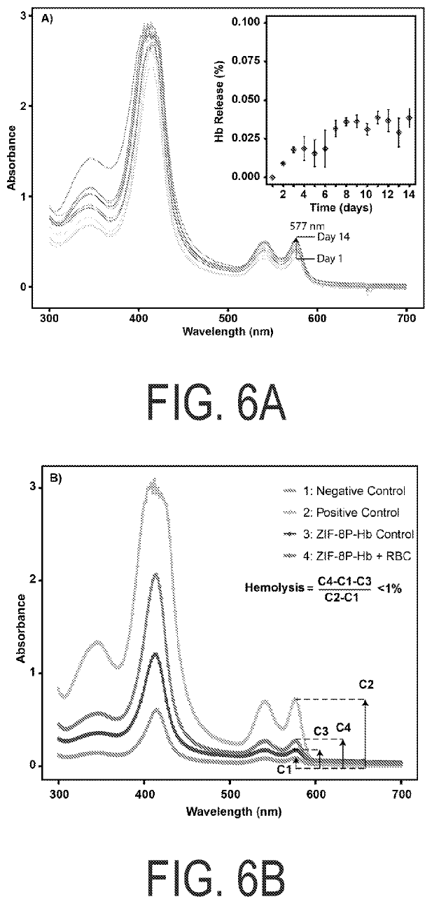

To measure the long-term stability of ZIF-8P-Hb nanoparticles, bHb leakage was evaluated at 4° C. for 14 days. In FIG. 6A, it was found that there was a gradual release of bHb in the first 7 days from 0.90±0.02% to 3.16±0.54%, which could be from the Hb trapped on the surface of ZIF-8P-Hb nanoparticles. From day 8 to 14, the bHb release curve plateaued, indicating no further leakage of bHb from the interior of the ZIF-8P-Hb nanoparticle. After 14 days, 3.85±0.63% bHb was released from the ZIF-8P-Hb nanoparticle, indicating its' superior stability.

### Hemocompatibility

Hemolysis is characterized by the rupture of RBCs and the release of cell-free Hb. Cell-free Hb can elicit renal failure, and tissue oxidative injury. Thus, it is important to measure the hemolytic activity of ZIF-8P-Hb nanoparticles as a guide to their potential biocompatibility with blood. In this study, hemolysis was assayed following 30 minutes incubation at 37° C. with RBCs. In FIG. 6B, negligible hemolysis (<5%) was observed for ZIF-8P-Hb nanoparticles, demonstrating favorable hemocompatibility. According to the ASTM E2524-08 standard, hemolysis >5% is considered significant.

### Conclusion

In general, the size of ZIF-8P-Hb nanoparticles was primarily controlled by Hmim:Zn molar ratio, flow rate during TFF processing, concentration of EDTA, and concentration of zinc nitrate. It was found that adding Hmim directly into the reaction vessel regulated particle size, which also affected the crystalline structure of the particle. Furthermore, we demonstrated that the high molar ratio of Hmim:Zn could be used to better control nucleation of ZIF-8P-Hb nanoparticles. The monodisperse size distribution was a result of the rapid nucleation rate facilitated by the relatively high molar ratio of Hmim:zinc, and TFF operated at relatively low flow rate did not exert a strong impact on the size distribution. Therefore, we established a scalable purification platform to manufacture ZIF-8 and ZIF-8P-Hb nanoparticles via TFF. The optimized synthesis protocol yielded relatively low batch-to-batch variance with respect to most biophysical properties including hydrodynamic diameter, zeta potential, oxygen equilibria, and oxygen offloading rate constant. The higher bHb loading and bHb encapsulation efficiency of ZIF-8P-Hb nanoparticles compared to prior attempts in the literature is critical in order to potentially use these materials to treat hemorrhagic shock. Future analysis of these materials will need to evaluate ZIF-8P-Hb nanoparticle efficacy and safety in vivo.

### REFERENCES

(1) Williams, A. T.; Lucas, A., Muller, C. R.; Munoz, C.; Bolden-Rush, C.; Palmer, A. F.; Cabrales, P. Resuscitation from Hemorrhagic Shock With Fresh and Stored Blood and Polymerized Hemoglobin. SHOCK 2020. https://doi.org/10.1097/shk.0000000000001530.
(2) Moore, E. E.; Moore, F. A., Fabian, T. C.; Bernard, A. C.; Fulda, G. J.; Hoyt, D. B.; Duane, T. M.; Weireter, L. J.; Gomez, G. A.; Cipolle, M. D.; Rodman, G. H.; Malangoni, M. A.; Hides, G. A.; Omert, L. A., Gould, S. A. Human Polymerized Hemoglobin for the Treatment of Hemorrhagic Shock When Blood Is Unavailable: The USA Multicenter Trial. J. Am. Coll. Surg. 2009, 208 (1), 1-13. https://doi.org/10.1016/j.jamcollsurg.2008.09.023.
(3) King, D. R.; Cohn, S. M.; Proctor, K. G.; Shackford, S. R.; Hall, J. R.; Aikawa, N., Malhotra, A. Resuscitation with a Hemoglobin-Based Oxygen Carrier after Traumatic Brain Injury. J. Trauma—Inj. Infect. Crit. Care 2005. https://doi.org/10.1097/01.ta.0000177711.52191.62.
(4) Stern, S.; Rice, J.; Philbin, N., McGwin, G.; Arnaud, F.; Johnson, T., Shannon Flournoy, W.; Ahlers, S.; Bruce Pearce, L.; McCarron, R.; Freilich, D. Resuscitation with the Hemoglobin-Based Oxygen Carrier, Hboc-201, in a Swine Model of Severe Uncontrolled Hemorrhage and Traumatic Brain Injury. Shock 2009 https://doi.org/10.1097/SHK.0b013e3181778dc3.
(5) Muller, C. R.; Courelli, V.; Lucas, A.; Williams, A. T.; Li, J. B.; Santos, F. Dos; Cuddington, C. T.; Moses, S. R.; Palmer, A. F.; Kistler, E. B.; Cabrales, P. Resuscitation from Hemorrhagic Shock after Traumatic Brain Injury with Polymerized Hemoglobin. Sci. Rep. 2021, 1-11. https://doi.org/10.1038/s41598-021-81717-3.
(6) Yu, M.; Han, J.; Dai, M.; Cui, P.; Li, H., Liu, Q. Influence of PEG-Conjugated Hemoglobin on Tumor Oxygenation and Response to Chemotherapy Influence of PEG-Conjugated Hemoglobin on Tumor. 2009, 1199. https://doi.org/10.1080/10731190802556674.
(7) Qu, J.; Guo, X.; Li, W.; Hou, W.; Zhang, H.; Luo, L.; Zhu, C.; Yang, J.; Yin, X.; Tao, Y.; Du, Y.; Lou, Y.; Chen, D.; You, J. Colloids and Surfaces B: Biointerfaces Preparation of Artificial Red Cell and Its Application on Alleviation of Tumor Hypoxia. Colloids Surfaces B Biointerfaces 2017, 160, 446-454. https://doi.org/10.1016/j.colsurfb.2017.09.039.
(8) Belcher, D. A., Lucas, A.; Cabrales, P.; Palmer, A. F. Polymerized Human Hemoglobin Facilitated Modulation of Tumor Oxygenation Is Dependent on Tumor Oxygenation Status and Oxygen Affinity of the Hemoglobin-Based Oxygen Carrier. Sci. Rep. 2020, No. 0123456789, 1-12. https://doi.org/10.1038/s41598-020-68190-0.
(9) Cabrales, P.; Sun, G.; Zhou, Y.; Harris, D. R., Tsai, A. G.; Intaglietta, M.; Palmer, A. F. Effects of the Molecular Mass of Tense-State Polymerized Bovine Hemoglobin on Blood Pressure and Vasoconstriction. J. Appl. Physiol. 2009, 107 (5), 1548-1558. https://doi.org/10.1152/japplphysiol.00622.2009.
(10) Baek, J. H.; Zhou, Y.; Harris, D. R.; Schaer, D. J.; Palmer, A. F.; Buehler, P. W. Down Selection of Polymerized Bovine Hemoglobins for Use as Oxygen Releasing Therapeutics in a Guinea Pig Model. Toxicol. Sci. 2012, 127 (2), 567-581. https://doi.org/10.1093/toxsci/kfs109.
(11) Gu, X.; Bolden-Rush, C.; Cuddington, C. T.; Belcher, D. A.; Savla, C.; Pires, I. S.; Palmer, A. F. Comprehensive Characterization of Tense and Relaxed Quaternary State Glutaraldehyde Polymerized Bovine Hemoglobin as a Function of Cross-Link Density. Biotechnol. Bioeng. 2020. https://doi.org/10.1002/bit.27382.
(12) Jahr, J. S.; Moallempour, M.; Lim, J. C. HBOC-201, Hemoglobin Glutamer-250 (Bovine), Hemopure (Registered Trademark) (Biopure Corporation). Expert Opin. Biol. Ther. 2008, 8 (9), 1425-1433,
(13) Vandegriff, K. D.; Young, M. A.; Keipert, P. E.; Winslow, R. M. The Safety Profile of Hemospan®: A New Oxygen Therapeutic Designed Using Maleimide Poly (Ethylene) Glycol Conjugation to Human Hemoglobin. Transfus. Altern. Transfus. Med. 2007, 9 (4), 213-225. https://doi.org/10.1111/j.1778-428X.2007.00083.x.
(14) Portoro, I.; Kocsis, L., Hermán, P.; Caccia, D., Perrella, M., Ronda, L., Bruno, S.; Bettati, S.; Micalella, C., Mozzarelli, A.; Varga, A.; Vas, M.; Lowe, K. C.; Eke, A. Towards a Novel Haemoglobin-Based Oxygen Carrier: Euro-PEG-Hb, Physico-Chemical Properties, Vasoactivity and Renal Filtration. Biochim. Biophys. Acta—Proteins Proteomics 2008. https://doi.org/10.1016/j.bbapap.2008.03.005.
(15) Usuba, A.; Osuka, F.; Kimura, T.; Sato, R.; Fujita, Y.; Yamashita, M.; Hoshino, C. Liposome Encapsulated Hemoglobin as a Resuscitation Fluid for Hemorrhagic Shock. Artif. Organs 1998, 22 (2), 116-122.
(16) Rameez, S.; Palmer, A. F. Simple Method for Preparing Poly (Ethylene Glycol)-Surface-Conjugated Liposome-Encapsulated Hemoglobins: Physicochemical Properties, Long-Term Storage Stability, and Their Reactions with O2, CO, and NO. Langmuir 2011, 27 (14), 8829-8840. https://doi.org/10.1021/la201246m.
(17) Belcher, D. A.; Cuddington, C. T.; Martindale, E. L.; Pires, I. S.; Palmer, A. F. Controlled Polymerization and Ultrafiltration Increase the Consistency of Polymerized Hemoglobin for Use as an Oxygen Carrier. Bioconjug. Chem. 2020. https://doi.org/10.1021/acs.bioconjchem.9b00766.
(18) Abid, H. R.; Tian, H.; Ang, H. M.; Tade, M. O.; Buckley, C. E.; Wang, S. Nanosize Zr-Metal Organic Framework (UiO-66) for Hydrogen and Carbon Dioxide Storage. Chem. Eng. J. 2012, 187, 415-420. https://doi.org/10.1016/j.cej.2012.01.104.
(19) Grant Glover, T.; Peterson, G. W.; Schindler, B. J.; Britt, D.; Yaghi, O. MOF-74 Building Unit Has a Direct Impact on Toxic Gas Adsorption. Chem. Eng. Sci. 2011, 66 (2), 163-170. https://doi.org/10.1016/j.ces.2010.10.002.
(20) Verdegaal, W. M.; Wang, K.; Sculley, J. P.; Wriedt, M.; Zhou, H. C. Evaluation of Metal-Organic Frameworks and Porous Polymer Networks for CO2-Capture Applications. ChemSusChem 2016, 9 (6), 636-643. https://doi.org/10.1002/cssc.201501464.
(21) Gross, A. F.; Sherman, E.; Vajo, J. J. Aqueous Room Temperature Synthesis of Cobalt and Zinc Sodalite Zeolitic Imidizolate Frameworks. Dalt. Trans. 2012, 47 (18), 5458-5460. https://doi.org/10.1039/c2dt30174a.
(22) Park, K. S.; Ni, Z.; Côté, A. P.; Choi, J. Y.; Huang, R.; Uribe-Romo, F. J.; Chae, H. K.; O'Keeffe, M.; Yaghi, O. M. Exceptional Chemical and Thermal Stability of Zeolitic Imidazolate Frameworks. Proc. Natl. Acad. Sci. U.S.A 2006, 103 (27), 10186-10191. https://doi.org/10.1073/pnas.0602439103.
(23) Luzuriaga, M. A.; Benjamin, C. E.; Gaertner, M. W.; Lee, H.; Herbert, F. C.; Mallick, S.; Gassensmith, J. J. ZIF-8 Degrades in Cell Media, Serum, and Some—but Not All—Common Laboratory Buffers. Supramol. Chem. 2019, 31 (8), 485-490.
(24) Ma, W.; Jiang, Q.; Yu, P.; Yang, L.; Mao, L. Zeolitic Imidazolate Framework-Based Electrochemical Biosensor for in Vivo Electrochemical Measurements. Anal. Chem. 2013, 85 (15), 7550-7557. https://doi.org/10.1021/ac401576u.
(25) Kaur, H.; Mohanta, G. C.; Gupta, V.; Kukkar, D.; Tyagi, S. Synthesis and Characterization of ZIF-8 Nanoparticles for Controlled Release of 6-Mercaptopurine Drug. J. Drug Deliv. Sci. Technol. 2017, 47, 106-112. https://doi.org/10.1016/j.jddst.2017.07.004.
(26) Peng, S.; Liu, J.; Qin, Y.; Wang, H.; Cao, B.; Lu, L., Yu, X. Metal-Organic Framework Encapsulating Hemoglobin as a High-Stable and Long-Circulating Oxygen Carriers to Treat Hemorrhagic Shock. ACS Appl. Mater. Interfaces 2019, 11 (39), 35604-35612. https://doi.org/10.1021/acsami.9b15037.
(27) Shah, N. K.; Ivone, R.; Shen, J.; Meenach, S. A. A Comparison of Centrifugation and Tangential Flow Filtration for Nanoparticle Purification: A Case Study on Acetalated Dextran Nanoparticles. Particuology 2020, 50, 189-196. https://doi.org/10.1016/j.partic.2019.06.004.
(28) Palmer, A. F.; Sun, G.; Harris, D. R. Tangential Flow Filtration of Hemoglobin. Biotechnol. Prog. 2009, 25 (1), 189-199. https://doi.org/10.1002/btpr.119.
(29) HILL, A. The Possible Effects of the Aggregation of the Molecules of Haemoglobin on Its Dissociation Curves. J Physiol (Lond). January 1910, pp 4-7.
(30) Meng, F.; Kassa, T.; Jana, S.; Wood, F.; Zhang, X.; Jia, Y.; D′ Agnillo, F.; Alayash, A. I. Comprehensive Biochemical and Biophysical Characterization of Hemoglobin-Based Oxygen Carrier Therapeutics: All HBOCs Are Not Created Equally. Bioconjug. Chem. 2018, 29 (5), 1560-1575. https://doi.org/10.1021/acs.bioconjchem.8b00093.
(31) Dirk De Korte, Mya Kleine, Herbert G. H. Korsten, A. J. V. Prolonged Maintenance of 2,3-Diphosphoglycerate Acid and Adenosine Triphosphate in Red Blood Cells during Storage. Transfusion 2008, 48 (6), 1081-1089. https://doi.org/10.1111/j.1537-2995.2008.01689.x.
(32) Drabkin, DL; Austin, J. Spectrophotometric Studies II. Preparations from Washed Blood Cells; Nitric Oxide Hemoglobin and Sulfhemoglobin. J. Biol. Chem. 1935, 112 (1), 51-65. https://doi.org/0021-9258.
(33) Luzuriaga, M. A.; Benjamin, C. E.; Gaertner, M. W.; Lee, H.; Herbert, F. C.; Mallick, S.; Gassensmith, J. J. ZIF-8 Degrades in Cell Media, Serum, and Some—but Not All—Common Laboratory Buffers. Supramol. Chem. 2019, 31 (8), 485-490. https://doi.org/10.1080/10610278.2019.1616089.
(34) Naito, Y.; Sakai, H.; Inoue, S.; Kawaguchi, M. Hemoglobin Vesicles Prolong the Time to Circulatory Collapse in Rats during Apnea. BMC Anesthesiol. 2017, 17 (1), 1-8. https://doi.org/10.1186/s12871-017-0338-y.
(35) Kaur, H.; Mohanta, G. C.; Gupta, V.; Kukkar, D.; Tyagi, S. Synthesis and Characterization of ZIF-8 Nanoparticles for Controlled Release of 6-Mercaptopurine Drug. J. Drug Deliv. Sci. Technol. 2017, 41, 106-112. https://doi.org/10.1016/j.jddst.2017.07.004.
(36) Liu, D., Mori, A.; Huang, L. Role of Liposome Size and RES Blockade in Controlling Biodistribution and Tumor Uptake of GM1-Containing Liposomes. BBA—Biomembr. 1992, 1104 (1), 95-101. https://doi.org/10.1016/0005-2736 (92) 90136-A.
(37) Zhou, Y. Synthesis and Biophysical Characterization of Polymerized Hemoglobin Dispersions of Varying Size and Oxygen Affinity as Potential Oxygen Carriers for Use in Transfusion Medicine. ProQuest Diss. Theses 2011, Ph.D.
(38) Umut, E. Surface Modification of Nanoparticles Used in Biomedical Applications. In Modern Surface Engineering Treatments; InTech, 2013. https://doi.org/10.5772/55746.
(39) Lv, B.; Wang, C.; Hou, J.; Wang, P.; Miao, L.; Li, Y.; Ao, Y.; Yang, Y.; You, G.; Xu, Y. Influence of Shear Forces on the Aggregation and Sedimentation Behavior of Cerium Dioxide (CeO2) Nanoparticles under Different Hydrochemical Conditions. J. Nanoparticle Res. 2016, 18 (7). https://doi.org/10.1007/s11051-016-3501-3.
(40) Jian, M.; Liu, B.; Liu, R.; Qu, J.; Wang, H.; Zhang, X. Water-Based Synthesis of Zeolitic Imidazolate Framework-8 with High Morphology Level at Room Temperature. RSC Adv. 2015, 5 (60), 48433-48441. https://doi.org/10.1039/c5ra04033 g.
(41) Cravillon, J.; Schröder, C. A.; Nayuk, R.; Gummel, J.; Huber, K., Wiebcke, M. Fast Nucleation and Growth of ZIF-8 Nanocrystals Monitored by Time-Resolved in Situ Small-Angle and Wide-Angle X-Ray Scattering. Angew. Chemie—Int. Ed. 2011, 50 (35), 8067-8071. https://doi.org/10.1002/anie.201102071.
(42) Kure, T.; Sakai, H. Preparation of Artificial Red Blood Cells (Hemoglobin Vesicles) Using the Rotation-Revolution Mixer for High Encapsulation Efficiency. ACS Biomater. Sci. Eng. 2021, 7 (6), 2835-2844. https://doi.org/10.1021/acsbiomaterials.1c00424.

### Example 2. ZIF-8 Metal Organic Framework Nanoparticle Loaded with Tense Quaternary State Polymerized Bovine Hemoglobin: Potential Red Blood Cell Substitute with Antioxidant Properties

### Overview

Due to several limitations associated with blood transfusion, such as the relatively short shelf life of stored blood, low risks of acute immune hemolytic reactions and graft-versus-host disease, many strategies have been developed to synthesize hemoglobin-based oxygen carriers (HBOCs) as universal red blood cell (RBC) substitutes. Recently, zeolite imidazole framework-8 (ZIF-8), a metal-organic framework, has attracted considerable attention as a protective scaffold for encapsulation of hemoglobin (Hb). Despite the exceptional thermal and chemical stability of ZIF-8, the major impediments to implementing ZIF-8 for Hb encapsulation are the structural distortions associated with loading large quantities of Hb in the scaffold as the Hb molecule has a larger hydrodynamic diameter than the pore size of ZIF-8. Therefore, to reduce the structural distortion caused by Hb encapsulation, we established and optimized a continuous-injection method to synthesize nanoparticle (NP) encapsulated polymerized bovine Hb (PolybHb) using ZIF-8 precursors (ZIF-8P-PolybHb NPs). The synthesis method was further modified by adding EDTA as a chelation-assisted etching agent, which reduced the ZIF-8P-PolybHb NP size to <300 nm. ZIF-8P-PolybHb NPs exhibited low oxygen affinity (36.4+3.2 mm Hg) compared to unmodified bovine Hb but was similar in magnitude to unencapsulated PolybHb. The use of the chemical cross-linker glutaraldehyde to polymerize bovine Hb resulted in the low Hill coefficient of PolybHb, indicating loss of Hb's oxygen binding cooperativity, which could be a limitation when using PolybHb as an oxygen carrier for encapsulation inside the ZIF-8 matrix. ZIF-8P-PolybHb NPs exhibited slower oxygen offloading kinetics compared to unencapsulated PolybHb, demonstrating successful encapsulation of PolybHb. ZIF-8P-PolybHb NPs also exhibited favorable antioxidant properties when exposed to H₂O₂. Incorporation of PolybHb into the ZIF-8 scaffold resulted in improved cytotoxicity in human umbilical vein endothelial cells compared to unloaded ZIF-8 NPs and ZIF-8 NPs loaded with bovine Hb. We envisage that such a monodisperse and biocompatible HBOC with low oxygen affinity and antioxidant properties may broaden its use as an RBC substitute.

### Introduction

Red blood cell (RBC) transfusion is a routine medical procedure to treat patients suffering from substantial surgical or traumatic blood loss; however, the relatively short ex vivo shelf-life of blood (42 days) and low probability risks of contracting unknown blood borne pathogens are the major concerns associated with RBC transfusion. To address these concerns, hemoglobin-based oxygen carriers (HBOCs) have been developed and tested in clinical trials as RBC substitutes over the last several decades. Unfortunately, none of these commercial HBOCs received FDA approval for clinical use in the U.S. due to severe adverse events observed during Phase III clinical trials. For instance, the presence of extracellular hemoglobin (Hb) and low molecular weight species (<300 kDa) in previous generations of HBOCs elicited vasoconstriction, systemic hypertension, myocardial infarction, acute renal damage, and heart lesions. This provides the rationale for the development of safer HBOCs.

Metal organic frameworks (MOFs) recently emerged as an excellent protective material to encapsulate biomolecules, cells, and DNA due to their superior solvothermal stability. ZIF-8, as a subclass of MOFs, is comprised of tetrahedral Zn²⁺ coordinated to 2-methlyimidazolate (Hmim) arranged in porous structures. Its remarkably high surface area and large pore size could potentially favor oxygen (O₂) storage and delivery. ZIF-8-ncapsulated Hb (ZIF-8@Hb) can possess extended circulation times in a murine model when compared to cell-free Hb. Additionally, ZIF-8's high tolerance towards basic environments, oxidation and high temperature can also be found to be present in ZIF-8@Hb. Hb can be encapsulated inside nanoparticles (NPs) using a porous coordination network (PCN)-333 (Al) of Al³⁺ connected by the organic linker triazine-2,4,6-triyl-tribenzoic acid. The resultant MOF-NPs encapsulating Hb can bind and release O₂.

Overall, MOF-encapsulated Hbs prepared to date exhibits relatively low Hb loading (<1 mg/mL), which limits their use as an RBC substitute. To address that issue, we developed ZIF-8 encapsulated bovine Hb NPs (ZIF-8P-Hb NPs) via synthesis at an ultrahigh molar ratio of Hmim:Hb, which facilitates the nucleation reaction. The resulting ZIF-8P-Hb NPs exhibited high structural stability, ultrahigh Hb encapsulation efficiency and loading capacity, and a monodisperse size distribution. However, we observed an increased O₂ affinity for ZIF-8P-Hb NPs when compared to cell-free Hb. In addition, HBOCs with relatively low O₂ affinity were found to be more favorable at delivering O₂ to surrounding tissues. Therefore, in this Example, we describe the encapsulation of tense quaternary state (T-state) polymerized bovine Hb (T-state PolybHb) into ZIF-8 precursors with the aim of designing a ZIF-8 HBOC with low O₂ affinity. T-state PolybHb was able to retain its low O₂ affinity by cross-linking bHb in the T-state with the chemical cross-linker glutaraldehyde. We also established and optimized a continuous-injection method coupled with a chelation-assisted chemical etching process to synthesize ZIF-8P-PolybHb NPs. ZIF-8P-PolybHb NPs exhibited a diameter of 170.9±17.4 nm and relatively narrow size distribution (PDI=0.042±0.011). ZIF-8P-PolybHb NPs had a significantly lower O₂ affinity (36.4+3.2 mm Hg) in comparison to ZIF-8P-Hb NPs (P₅₀=11.8 mm Hg). Additionally. ZIF-8P-PolybHb NPs exhibited a more negative zeta potential than ZIF-8P-Hb NPs and prior generations of MOF-Hb NPs, demonstrating potentially improved biocompatibility. Favorable antioxidant (H₂O₂) properties and hemocompatibility (<5% hemolysis of RBCs) were also observed for ZIF-8P-PolybHb NPs. Therefore, encapsulation of T-state PolybHb using ZIF-8 precursors should not only be able to facilitate tissue O₂ offloading, but also enhance biocompatibility.

### Materials and Methods

### Bovine Hb Purification.

Bovine Hb (bHb) was purified from sodium citrate anticoagulated whole blood (Quad Five, Ryegate, MT) via TFF. Two HF cartridges with MWCOs of 500 and 50 kDa (Repligen Corporation, Rancho Dominguez, CA) were used to purify and concentrate bHb. The final product was stored at >200 mg/mL in the freezer (−80° C.) for future use.

### bHb Polymerization and PolybHb Purification.

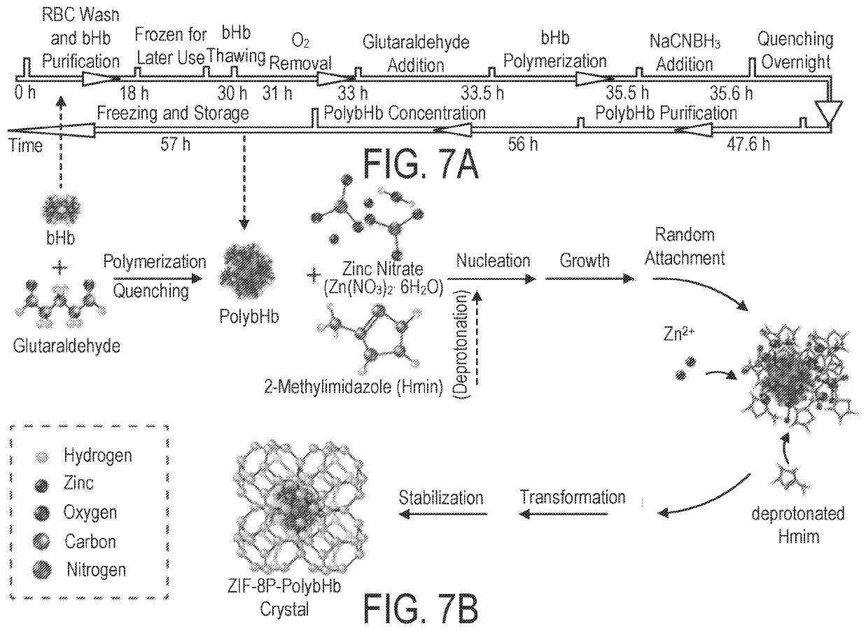

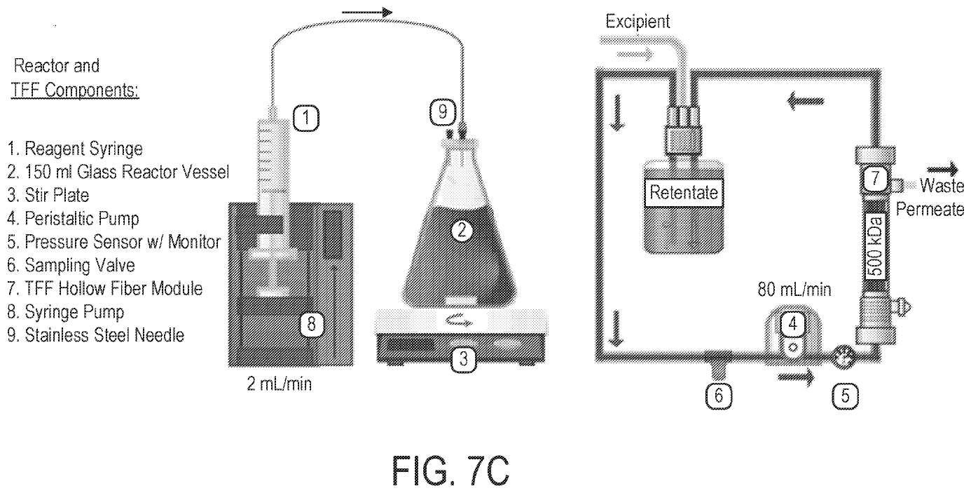

Glutaraldehyde was used as the chemical cross-linker to synthesize tense quaternary state (T-state) polymerized bHb (PolybHb). FIG. 7A shows a schematic of the PolybHb synthesis and purification process. Frozen bHb was quickly thawed in a water bath at 37° C. The thawed bHb solution was diluted to ˜20 mg/ml in phosphate buffered saline (PBS, 0.1 M, pH 7.40) and then transferred into a 2 L reactor vessel. PolybHb was synthesized at a 25:1 molar ratio of glutaraldehyde to bHb in the T-state. Complete deoxygenation of the bHb solution was accomplished via a 3-M MiniModule gas/liquid exchange module (Maplewood) with nitrogen as the sweep gas. Sodium dithionite (Na₂S₂O₄) was added to the vessel to deoxygenate the bulk solution (pO₂˜0.0 mmHg). Glutaraldehyde was prepared in PBS at a total volume of 50 mL and added to the bHb solution at a flow rate of 2 mL/min. After glutaraldehyde addition (˜30 minutes), the mixture was allowed to react for 2 hours at 37° C., chased by a bolus injection of NaCNBH₃ to quench the reaction as previously described in the literature.

After PolybHb synthesis, clarification, purification, and concentration of PolybHb was performed via a two-stage TFF process. Specifically, a 500 kDa hollow fiber (HF) module was used to clarify the PolybHb by removing the high MW species (>500 kDa). A 100 kDa TFF module was then used to retain and buffer exchange the low MW PolybHb (>100 kDa but <500 kDa) into PBS (0.1 M, pH 7.4) for subsequent encapsulation by ZIF-8 precursors.

### Synthesis and Purification of ZIF-8-PolybHb NPs.

FIG. 7B shows the synthesis scheme for NP encapsulated PolybHb using ZIF-8 precursors (ZIF-8P-PolybHb NPs). Initially, 25 mL of deionized (DI) water was used to fully dissolve 100 mg of Zn(NO₃)₂·6H₂O. An equal volume of DI water was used to dissolve 827.9 mg 2-methylimidazole (Hmim) in a 150 mL Erlenmeyer flask. 250 mg PolybHb was then added into the Hmim solution with continuous stirring (500 rpm) for 5 min. The Zn(NO₃)₂·6H₂O solution was then transferred into a 50 ml syringe and slowly added into the Hmim solution at a flow rate of 2 mL/min. The reaction was allowed to proceed for 1 hour at 25° C., and chased by a bolus addition of 4 mL EDTA (80 mM, pH 8.0). EDTA (chelating agent) was used to selectively etch the ZIF-8-PolybHb NPs via binding to the metal ions (Zn²⁺), which facilitates the breakage of the coordination bond (Zn²⁺-Hmim) and slows down the growth of the ZIF-8 NPs. The solution was then placed in the refrigerator (4° C.) for overnight aging. The resulted ZIF-8P-PolybHb NPs were purified by washing with TFF using a 500 kDa mPES HF cartridge first with DI water (for 2 diacycles) and subsequently buffered exchanged into PBS (0.1 M, pH 7.4) for another 6˜10 diacycles. The final product was stored at 4° C. for further use. Bare ZIF-8 NPs were synthesized using the procedure described in Example 1.

### Electron Microscopy and X-Ray Diffraction Analysis.

The morphology of ZIF-8, ZIF-8P-Hb and ZIF-8P-PolybHb NPs was imaged via a FEI Tecnai G2 Biotwin transmission-electron microscope (Thermo Fisher Scientific). Protein samples were diluted to ˜0.5-1 mg/mL in DI water. XRD spectra were recorded on a Bruker D8 Advance diffractometer (AXS, Bruker, Germany) with Cu target from 5° to 45°.

### Hydrodynamic Diameter.

The hydrodynamic diameter of ZIF-8P-PolybHb NPs were measured via dynamic light scattering (DLS) analysis using a BI-200SM goniometer (Brookhaven Instruments Corp., Holtsville, NY) at an angle of 90° and wavelength of 637 nm. Protein samples were diluted to ˜0.5-1 mg/mL in DI water. The hydrodynamic diameter was calculated via the instrument software.

### Zeta (ζ) Potential.

The ζ potential of bHb, PolybHb, ZIF-8P-Hb and ZIF-8P-PolybHb was measured using a Brookhaven Instruments ZetaPals instrument (Holtsville, NY) at room temperature. All samples were diluted to ˜1 mg/ml in DI water.

### Electron Microscopy and X-ray Diffraction Analysis.

The morphology of ZIF-8, ZIF-8P-Hb and ZIF-8P-PolybHb NPs was imaged via a FEI Tecnai G2 Biotwin transmission-electron microscope (Thermo Fisher Scientific). Protein samples were diluted to ˜0.5-1 mg/mL in DI water. XRD spectra were recorded on a Bruker D8 Advance diffractometer (AXS, Bruker, Germany) with Cu target from 5° to 45°

### O₂ Equilibrium Analysis.

To study the O₂ equilibrium properties at ˜37° C., the O₂ equilibrium curves (OECs) for bHb, PolybHb, ZIF-8P-Hb and ZIF-8P-PolybHb were measured using a Hemox Analyzer (TCS Scientific Corp., New Hope, PA) in PBS (0.1 M, pH 7.4). The OEC was fit to the Hill equation to regress the O₂ binding affinity (P₅₀, partial pressure of O₂ (pO₂) at which the Hb is half saturated with 02) and Hill coefficient (n).

### Rapid O₂ Offloading Kinetics.

The (2 offloading kinetics of bHb, PolybHb, ZIF-8P-Hb NPs and ZIF-8P-PolybHb NPs was studied via rapidly mixing Na₂S₂O₄ solution (1.5 mg/mL) with protein samples (12.5 μM, heme basis) in a microvolume stopped-flow spectrophotometer (Applied Photophysics Ltd., Surrey, United Kingdom). The resultant absorbance change was monitored at 437.5 nm and fit to a mono-exponential function to regress the rate constant for O₂ offloading (k_{off,O2}).

### Haptoglobin Binding Kinetics.

The binding kinetics between human haptoglobin (Hp) and bHb or PolybHb or ZIF-8P-PolybHb NPs was monitored via stopped flow fluorescence spectrometry (λ_{excitation}=285 nm, λₑₘᵢₛₛᵢₒₙ=310 nm) in PBS (0.1 M, pH 7.4). The pseudo first order Hp binding rate constant was calculated by fitting the fluorescence intensity to a mono-exponential equation. The pseudo first order rate constants were then linearly regressed as a function of Hb concentration to obtain the bimolecular rate constant.

### Auto-Oxidation Kinetics.

All samples including bHb, PolybHb, and ZIF-8P-PolybHb NPs were first diluted to 0.31 mM (heme basis) in PBS (0.1 M, pH 7.4) and submerged in water bath at 37° C. for 24 hours. The spectra (300-700 nm) of each sample were collected via UV-visible spectrometry every 60 mins. The metHb concentration was measured using the cyanmethemoglobin method. The auto-oxidation rate constant was calculated by fitting the normalized Hb concentration (ln([Hb²⁺]/[Hb²⁺]₀; ([Hb²⁺], the concentration of oxyHb) to a linear function.

Hb Release from ZIF-8P-Hb NPs.

To study the hydrothermal stability of ZIF-8P-PolybHb NPs, PolybHb release from ZIF-8P-PolybHb NPs was recorded over 7 days at 37° C. in PBS (0.1 M, pH 7.4). The mass of PolybHb in the pooled permeate was calculated by measuring the PolybHb concentration using UV-visible spectrometry and measuring the total permeate volume after filtering the ZIF-8P-PolybHb NP solution through a 500 kDa mPES TFF cartridge.

### Hemocompatibility of ZIF-8P-PolybHb NPs.

Expired human RBCs and plasma units were generously donated by Transfusion Services, Wexner Medical Center, The Ohio State University (Columbus, OH). The hemolysis assay was performed using know methods. Briefly, 1 mL of human RBCs was mixed with 1 mL of ZIF-8P-PolybHb solution suspended in 0.9% saline solution. The mixtures were incubated at 37° C. in a water bath incubator for 30 minutes and then centrifuged to collect the supernatant. The Hb concentration of the supernatant was measured with UV-visible spectrometry. Hemolysis was determined by the ratio of the mass of Hb in the supernatant of the ZIF-8P-PolybHb/RBC mixture to the total mass of Hb derived from the RBCs.

### Total bHb and Methemoglobin (MetHb) Levels.

Total bHb and metHb concentrations were determined using the cyanmethemoglobin method. To accurately measure the bHb concentration and reduce the effect of NP scattering, the cyanmethemoglobin method was slightly modified by adding EDTA (80 mM) to fully dissolve the ZIF-8P-PolybHb NP crystalline structure prior to the addition of cyanide. Spectrophotometric absorbance measurements were obtained using a HP 8452A diode array spectrophotometer (Olis, Bogart, GA).

H₂O₂ Oxidation Kinetics.

To investigate the antioxidant properties of ZIF-8P-PolybHb NPs against H₂O₂, bHb, PolybHb, ZIF-8P-Hb and ZIF-8P-PolybHb NPs were exposed to an excessive amount of H₂O₂ (100 mM) at different concentrations of total Hb ([Hb]: 0.125, 0.25, 0.5 mg/mL). The change in fractional composition of hemichrome, oxyHb, and metHb was evaluated through spectral deconvolution via an open-source Python package Alchromy (www.alchromy.com).

### PolybHb Encapsulation Efficiency.

The encapsulation efficiency (EE %) of bHb inside ZIF-8P-PolybHb NPs was calculated using the equation below.

$$EE ( \% ) = ( \frac{H {b}_{init} - {Hb}_{per}}{H {b}_{init}} ) \times 100 \%$$

where Hbₚₑᵣ corresponds to the mass of PolybHb in the permeate solution at the end of the TFF washing process and Hbᵢₙᵢₜ corresponds to the initial mass of PolybHb.

### Cytotoxicity.

The cytotoxicity of ZIF-8-PolybHb NPs was assessed with an MTS assay using human umbilical vein endothelial cells (HUVECs). Prior to testing, cells were grown in human large vessel endothelial cell basal media supplemented with large vessel endothelial supplement and 1% penicillin-streptomycin until 80-90% confluence. Passages 2-4 were used for testing. Cells were seeded at 1×10⁵/mL in a 96-well plate and left to adhere for 24 hours before application of PolybHb, unloaded ZIF-8 NPs, ZIF-8-bHb NPs, and ZIF-8-PolybHb NPs at varying concentrations (0-500 μg/mL). Cells were incubated with treatment groups for 24 hours before staining with MTS reagent for 3 hours. Media and DMSO were used as a positive and negative control, respectively. The optical density at 490 nm was measured with a Varioskan™ Lux multimode microplate reader using SkanIT software (Thermo Fisher Scientific, Waltham, MA).

### Statistical Analysis.

In this Example, all statistical analysis was performed using a t-test, and a p value of <0.05 was considered significant.

TABLE 2
Summary of the biophysical properties of ZIF-8, ZIF-8P-Hb, and ZIF-8P-PolybHb in comparison to bHb, PolybHb,
HbV, and RBCs. The effect of PolybHb encapsulation using ZIF-8 precursors on the biophysical properties of
ZIF-8P-PolybHb NPs was studied by comparing the hydrodynamic diameter, zeta potential, oxygen equilibria, oxygen
offloading rate constant, and Hp binding rate constant to native bHb and other HBOCs from the literature.

| | | | | ZIF-8P- | | | |
|---|---|---|---|---|---|---|---|
| Parameter | bHb | ZIF-8 | ZIF-8P-Hb | PolybHb | PolybHb | HbV | RBCs |
| P₅₀ (mm Hg) | 26.3 ± 0.5 | — | 11.8 ± 0.6* | 36.4 ± 3.2* | 39.6 ± 2.3* | 32 | 25.6 |
| Cooperativity (n) | 2.50 ± 0.10 | — | 1.99 ± 0.08* | 1.07 ± 0.21* | 1.08 ± 0.03* | 2.5 | 2.8 |
| Average MW (kDa) | 65 | — | — | — | 216 | — | — |
| D_{eff} (nm) | 6.9 ± 0.3 | 90.3 ± 11.8* | 106.0 ± 9.7* | 170.9 ± 17.4* | 11.2 ± 1.5* | 251 ± 87 | 7-8 × 10³ |
| PDI | 0.1 | 0.065 ± 0.006 | 0.085 ± 0.008 | 0.042 ± 0.011 | 0.3 | — | — |
| Zeta potential (mV) | −5.8 ± 0.7 | 40.7 ± 2.0* | −11.2 ± 0.9* | 15.0 ± 2.3* | 17.1 ± 1.4* | −18 | −12.5 |
| k_{off, O2} (s⁻¹) | 45.86 ± 1.00 | — | 19.57 ± 1.27* | 16.96 ± 2.62* | 39.96 ± 3.45 | 9.4 | 5.1 |
| k_{Hb-Hp} (μM⁻¹s⁻¹) | 0.1491 | — | 0.0405 | 0.0064 | 0.0185 | — | — |
| k_{0X} (h⁻¹) | 0.0123 ± 0.0019 | — | 0.0066 ± 0.0007 | 0.0068 ± 0.0009 | 0.0068 ± 0.0005 | — | — |
| MetHb level (%) | 1.0 ± 0.3 | — | 4.1 ± 0.4* | 8.30 ± 0.6* | 5.5 ± 0.2* | <10 | <1 |

(*denotes statistical significance (p < 0.05) compared to bHb.

### Results and Discussion

### Crystalline Structure and Morphology.

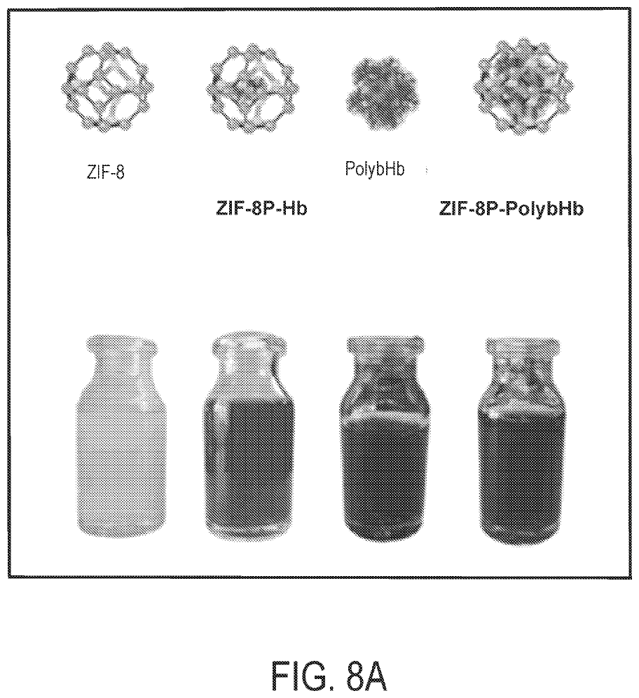

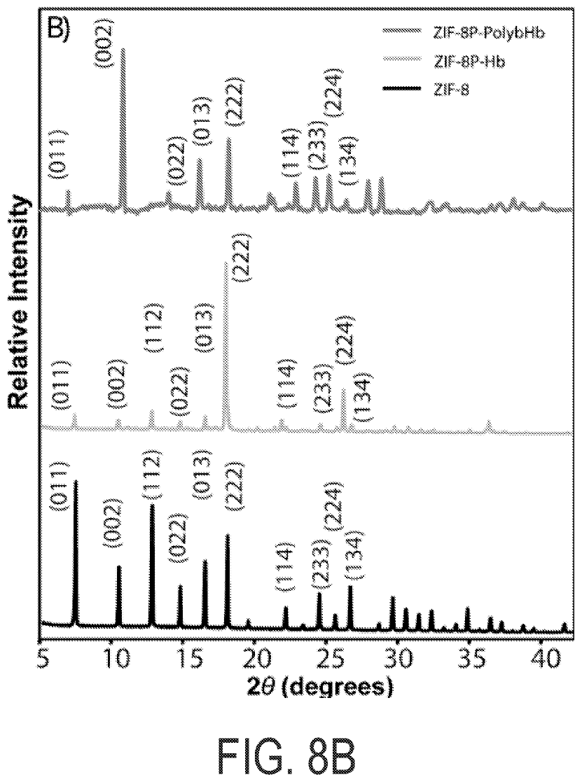

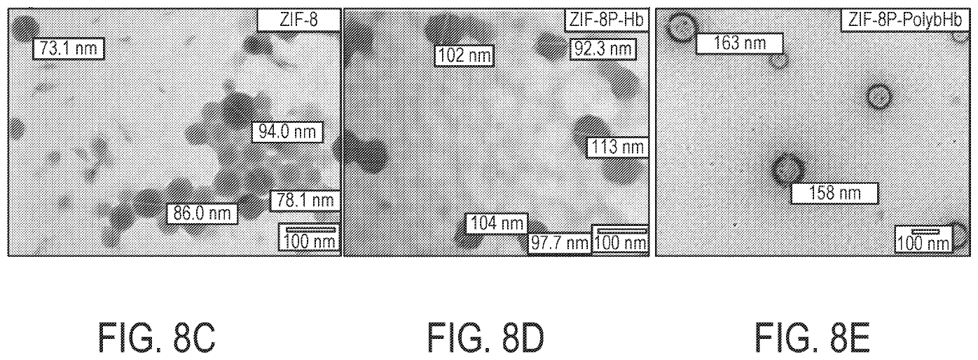

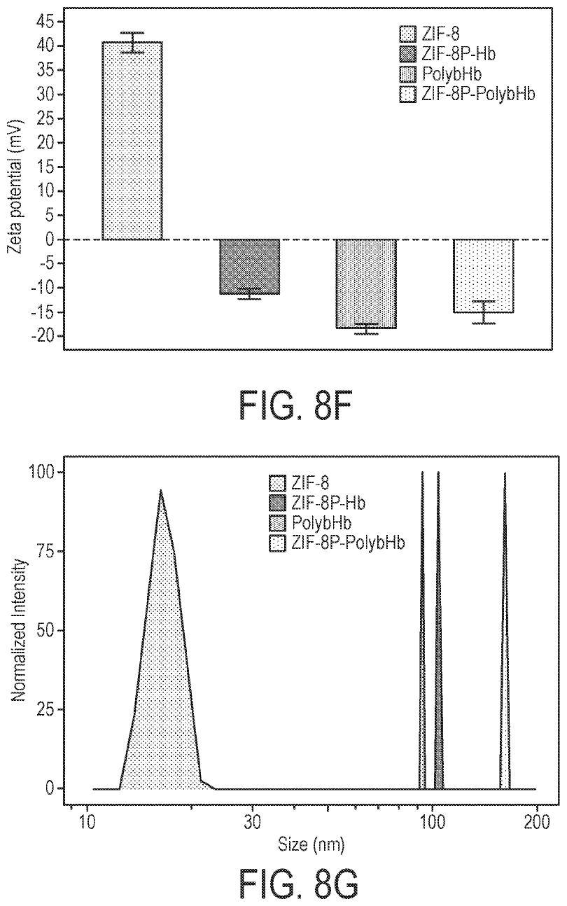

FIG. 8A (from left to right) shows images 5 of the synthesized ZIF-8 NP, ZIF-8P-Hb NP, PolybHb, and ZIF-8P-PolybHb NP solutions, which appeared white, light red, dark red, and crimson red in color, respectively. ZIF-8 NPS was synthesized as a control, and appeared milky white color. The slightly more turbid color of ZIF-8P-PolybHb NPs compared to PolybHb qualitatively demonstrates successful encapsulation of PolybHb and the formation of large particles. The color difference between ZIF-8P-Hb NPs and ZIF-8P-PolybHb NPs could be the result of the quaternary state of the 10 encapsulated PolybHb in ZIF-8P-PolybHb NPs versus the encapsulated bHb in ZIF-8P-Hb NPs. The XRD patterns of ZIF-8, ZIF-8P-Hb NPs, PolybHb, and ZIF-8P-PolybHb NPs are shown in FIG. 8B, whereas ZIF-8 NPs exhibited the typical ZIF-8 crystalline phases. ZIF-8P-Hb NPs was found to share similar peaks as ZIF-8 NPs, suggesting preservation of the ZIF-8 crystalline structure after encapsulation of bHb (5 mg/mL). Although ZIF-8P-Hb NPs contained a relatively low number of new phases, there was a significantly high diffraction intensity at 2θ=18.0°. This could be attributed to the residual Hmim that was trapped in the crystal cell and thus not completely removed during the TFF washing process, since we added the Hmim powder directly into the solution. From the XRD pattern of ZIF-8P-PolybHb NPs, a lower intensity at 2θ=18.0° suggests that dissolving both Zn(NO₃)₂ and Hmim as well as controlling the addition rate helps reduce the amount of Hmim trapped inside the ZIF-8 framework. Additionally, ZIF-8P-PolybHb NPs had peaks at structural planes of (011), (002), (022), (013), (222), (114), (233), and (134), which align with that of ZIF-8. However, the intensity at plane (112) was quite low likely due to the distortion of the ZIF-8 lattice when encapsulating larger proteins e.g., PolybHb (11.2±1.5 nm in diameter). Such distortions could also be reflected from the slightly left shifted peaks at plane (022), and (013). Both ZIF-8P-Hb NPs and ZIF-8P-PolybHb NPs were synthesized at the same concentration of bHb (5 mg/mL). Thus, the difference between the PXRD patterns of those materials should be a result of encapsulating proteins with different hydrodynamic diameters. TEM images in FIGS. 8C, 8D, and 8E reveal the morphology of ZIF-8 NPs, ZIF-8P-Hb NPs, and ZIF-8P-PolybHb NPs, respectively. Both ZIF-8 NPs and ZIF-8P-Hb NPs displayed a polyhedral shape. ZIF-8P PolybHb NPs exhibited a spherical shape, which is drastically different in comparison to both ZIF-8 NPs and ZIF-8P-Hb NPs, demonstrating successful completion of the chelation-mediated etching process.

### Size Distribution.

According to TEM analysis, ZIF-8 NPs, ZIF-8P-Hb NPs, and ZIF-8P-PolybHb NPs were found to have an average diameter of ˜80.53 nm, ˜102.33 nm, and ˜145.25 nm, which are close to the DLS results (FIG. 8G). From DLS analysis, ZIF-8 NPs, ZIF-8P-Hb NPs, and ZIF-8P-PolybHb NPs exhibited a hydrodynamic diameter of 90.3±11.8, 106.9±9.7, and 170.9±17.4 nm. Given that the size of NPs calculated from DLS is based on the Stoke-Einstein equation, direct visualization of the NPs via TEM should yield a more accurate particle diameter. From the TEM images in FIGS. 8C and 8D, both ZIF-8 NPs and ZIF-8P-Hb NPs exhibit polyhedral morphology due to the coordination between Zinc and Hmim. Since Zn²⁺ possesses sp³ hybrid orbitals, it forms a tetrahedral structure with Hmim, which is then be repeated to yield the ZIF-8 crystal structure. In comparison to ZIF-8 NPs and ZIF-8P-Hb NPs, ZIF-8P-PolybHb NPs exhibits a spherical shape instead of a polyhedral shape, which could be attributed to the chemical etching process. The chemical etching process not only “shaved off” the exterior edges of the NPs, but also created a hollow structure (FIG. 8E), which potentially enables larger protein loading due to the expanded cavities in the NPs. ZIF-8P-PolybHb NPs were also observed to have relatively narrow size distribution (PDI=0.042±0.011). Encapsulation of PolybHb using ZIF-8 precursors drastically narrowed the size distribution of ZIF-8P-PolybHb NPs (PDI=˜0.3) as shown in Table 2. Given that the size of ZIF-8P-PolybHb NPs are <300 nm, they should be less prone to clearance by the reticuloendothelial system (RES).

### Zeta Potential.

To study the effect of PolybHb encapsulation on the surface charge of ZIF-8P-PolybHb NPs, we measured the zeta potential of ZIF-8P-PolybHb NPs in comparison to ZIF-8 NPs, ZIF-8P-Hb NPs, and PolybHb. In FIG. 8F, bare ZIF-8 NPs were found to have a highly positive zeta potential of 40.7±2.04 mV. In comparison to ZIF-8 NPs, both ZIF-8P-Hb NPs and ZIF-8P-PolybHb NPs were observed to have a negative zeta potential. The reduction in zeta potential of ZIF-8 NPs is indicative of successful encapsulation of bHb and PolybHb by the ZIF-8 precursors, respectively. ZIF-8P-PolybHb NPs were found to have a slightly more negative zeta potential (−15.0±2.3 mV) in comparison to ZIF-8P-Hb NPs (−11.2±0.9 mV). This makes sense since PolybHb was observed to have a more negative zeta potential (−17.7±1.4 m V) than bHb (−5.8±0.7 mV). Due to its negatively charged surface, ZIF-8P-PolybHb NPs should be less prone to aggregation and potentially exhibit less cytotoxic than ZIF-8P-Hb NPs.

### Oxygen Affinity and Offloading Kinetics.

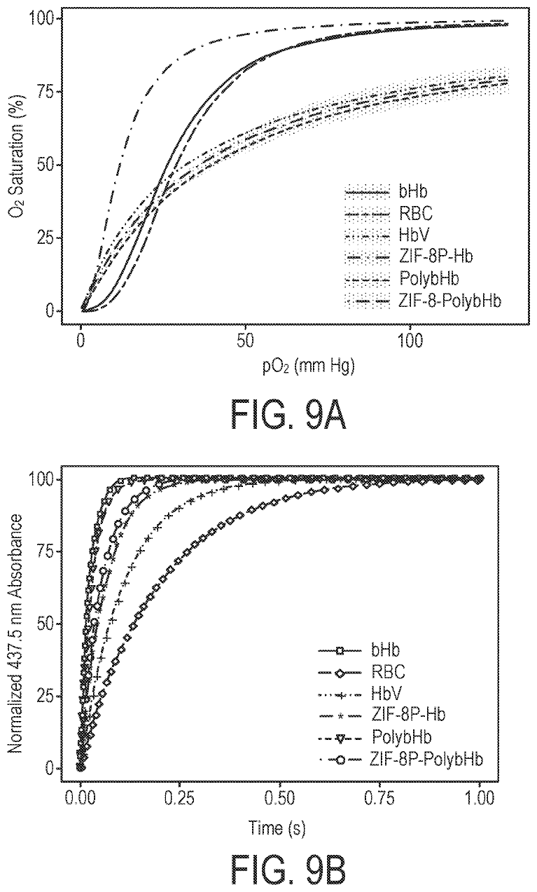

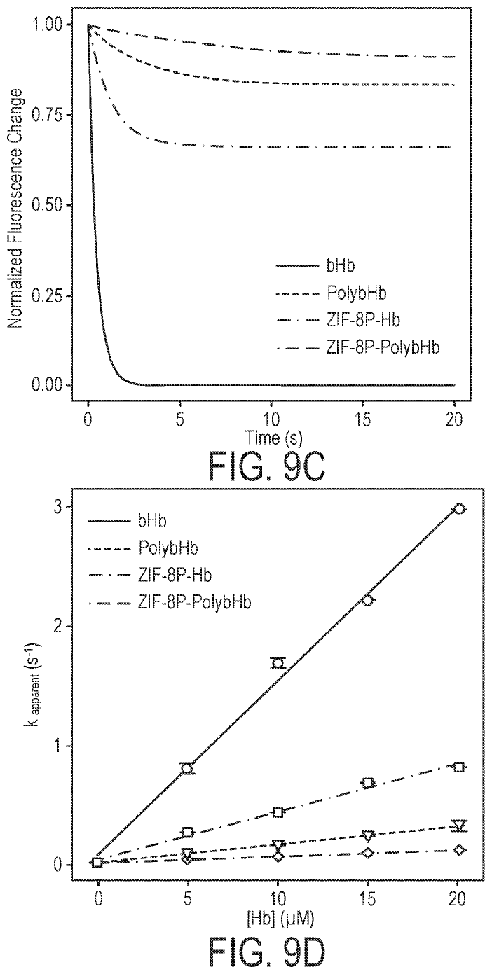

The O₂ affinity of PolybHb and ZIF-8P-PolybHb NPs was assessed by measuring the O₂ equilibrium curves (OECs), which were then fit to the Hill equation to regress the P₅₀ (partial pressure of O₂ at which Hb is half saturated with O₂) and n (Hill coefficient). In FIG. 9A, ZIF-8P-PolybHb NPs were found to exhibit a similar OEC when compared to its precursor, PolybHb. While for ZIF-8P-Hb NPs, there was a left shift in the OEC compared to cell-free bHb. We have previously reported that the hydrogen bond between the ZIF-8 framework and bHb could affect the O₂ equilibrium. However, for ZIF-8P-PolybHb NPs, such behavior disappears when we compare the P₅₀ of ZIF-8P-PolybHb NPs (36.4±3.2 mm Hg) to PolybHb (39.6±2.3 mm Hg). This could be a consequence of the intra/intermolecular crosslinking of bHb using glutaraldehyde, which locked the quaternary structure of PolybHb in the T-state. PolybHb is unable to shift from the T-state to the R-state, which allows more tolerance to the spatial structural changes elicited by the hydrogen bonds. The difference between the P₅₀ of ZIF-8P-Hb NPs and ZIF-8P-PolybHb NPs also demonstrates the ability to tune the O₂ binding properties of the NPs via modifying the quaternary state of the encapsulated materials. Intra/intermolecular crosslinking will also affect cooperative O₂ binding of ZIF-8P-PolybHb NPs, for which the Hill coefficient was found to be 1.07±0.21. Similar to PolybHb, ZIF-8P-PolybHb NPs also exhibited non-cooperative O₂ binding. In comparison to cell-free bHb (2.50±0.10), RBCs (˜2.8) and HbVs (˜2.5), the loss of O₂ binding cooperativity of ZIF-8P-PolybHb NPs is due to the chemical cross-linking in the polymerized bHb superstructure, which restricted conformational changes in the bHb tetramer thus reducing its Oz binding cooperativity.

In FIG. 9B, ZIF-8P-PolybHb NPs were found to have a significantly lower k_{off,O2} (16.96±2.62 s⁻¹) when compared to its precursor PolybHb (39.96±3.45 s⁻¹), likely a consequence of protein encapsulation by the ZIF-8 precursors, which results in an increased O₂ diffusion barrier, thus decreasing the K_{off,O2}. Interestingly, ZIF-8P-PolybHb NPs had a slightly lower k_{off,O2} (16.96±2.62 s⁻¹) in comparison to ZIF-8P-Hb (19.57±1.27 s⁻¹) despite the fact that ZIF-8P-PolybHb has a higher P₅₀ (36.4±3.2 mm Hg) than ZIF-8P-Hb NPs (11.2±0.9 mm Hg). This could be explained by the increased hydrodynamic diameter of ZIF-8P-PolybHb NPs when compared to ZIF-8P-Hb NPs which yields a higher O₂ diffusion barrier.

### Haptoglobin Binding Kinetics.

The pseudo first order Hp binding kinetics of bHb, PolybHb, ZIF-8P-Hb NPs, and ZIF-8P-PolybHb NPs are shown in FIG. 9C. ZIF-8P-PolybHb NPs did not show appreciable Hp binding when compared to PolybHb, demonstrating enhanced protection against encapsulated PolybHb recognition by plasma Hp. Additionally, a lower number of binding sites on Hp was quenched by ZIF-8P-PolybHb NPs in comparison to ZIF-8P-Hb NPs. The second order kinetic rate constant is shown in FIG. 9D. ZIF-8P-PolybHb NPs were found to have a significantly lower k_{Hp-Hb} (0.0064 μM⁻¹ s⁻¹) in comparison to ZIF-8P-Hb NPs (0.0405 μM⁻¹ s⁻¹). This could be attributed to the difference between the Hp binding rate of their precursors i.e., PolybHb (0.0185 μM⁻¹ s⁻¹) and bHb (0.1491 μM⁻¹ s⁻¹). Overall, ZIF-8P-PolyHb NPs were found to have a three-fold lower k_{Hp-Hb} (0.0064 μM⁻¹ s⁻¹) compared to PolybHb (0.0185 μM⁻¹ s⁻¹).

### Auto-Oxidation Kinetics.

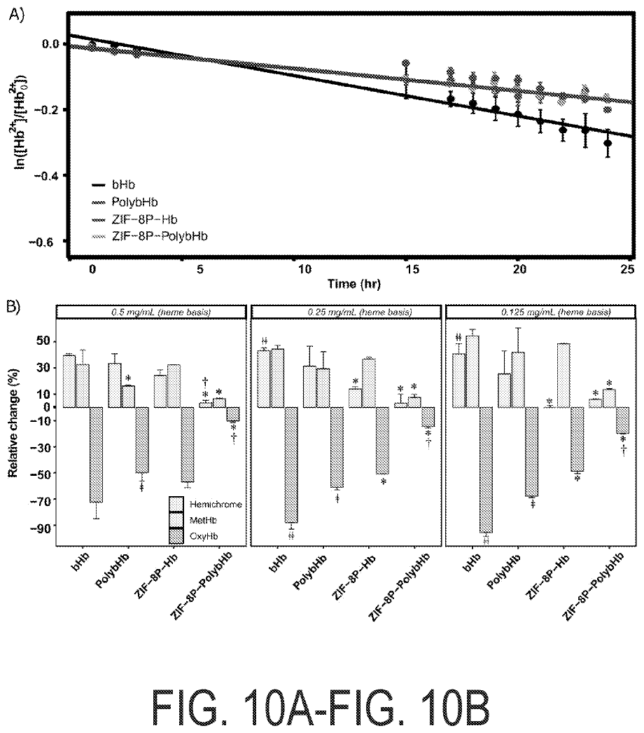

The auto-oxidation kinetics of the materials under physiological conditions was monitored by measuring the metHb level of bHb, PolybHb, and ZIF-8P-PolybHb NPs in PBS (0.1 M, pH 7.4) at 37° C. over a 24-hr period. The metHb level was assessed via the cyanmethemoglobin method. In FIG. 10A, low MW T-state PolybHb ([GA:Hb]=25:1) exhibited a lower auto-oxidation rate constant compared to unmodified Hb. Both bHb and T-state low MW (LMW) PolybHbs displayed monophasic kinetics. ZIF-8P-PolybHb NPs (0.0068±0.0009 h⁻¹) had a similar auto-oxidation rate constant compared to unencapsulated PolybHb (0.0068±0.0005 h⁻¹), p>0.05. In comparison to bHb (0.0123=0.0019 h⁻¹), the auto-oxidation rate constant of ZIF-8P-Hb NPs (0.0066±0.0007 h⁻¹) was not significantly different, p>0.05. Therefore, encapsulation of PolybHb/bHb with ZIF-8 precursors did not deteriorate the hydrothermal stability of encapsulated PolybHb/bHb.

H₂O₂ Oxidation Kinetics.

Reactive oxygen species (ROS) including singlet oxygen (¹O₂), hydroxyl radical (⁻OH), peroxyl radical (⁻OOR), superoxide (⁻O₂⁻), and hydrogen peroxide (H₂O₂) are generated from O₂ mainly inside the mitochondria during cellular metabolism. Endogenous H₂O₂ is known to cause oxidative damage of cellular proteins e.g., Hb, which would be even more extensive for cell-free Hb due to the lack of catalase. To investigate the antioxidant properties of ZIF-8P-PolybHb NPs against H₂O₂, bHb, PolybHb, and ZIF-8P-PolybHb NPs were exposed to H₂O₂ at different concentrations ([Hb], 0.125, 0.25, 0.5 mg/mL). FIG. 10B reveals significant differences between the antioxidant properties of bHb, PolybHb, and ZIF-8P-PolybHb NPs. Specifically, 72.34±12.69% of bHb (0.5 mg/mL) was converted to hemichrome (39.55±1.55%) and metHb (32.79±11.14%) after incubation in excess H₂O₂ for 5 mins, whereas more than 95% of bHb was converted to hemichrome and metHb at lower concentrations (˜0.125 mg/mL). In comparison to bHb, ZIF-8P-Hb NPs was found to yield 14.08±1.72% of hemichrome, which is significantly lower than that of bHb (39.55±1.55%) at 0.25 mg/mL [Hb]. A similar trend was observed when compared to hemichrome formation at 0.125 mg/mL [Hb], demonstrating improved protection against H₂O₂ due to encapsulation using ZIF-8 precursors. PolybHb was observed to have a higher tolerance against H₂O₂ oxidation compared to bHb. For example, ˜49.93±6.42% of PolybHb was converted to hemichrome (33.58±7.31%) and metHb (16.38±0.90%), which was significantly less than bHb. This could be attributed to the higher diffusion barrier for H₂O₂ to diffuse inside the PolybHb superstructure when we increased the hydrodynamic diameter of bHb via chemical cross-linking. Such phenomena should be observed in the PolybHb solutions at all concentrations (0.125, 0.25, and 0.5 mg/mL). Interestingly, after encapsulation of PolybHb using ZIF-8 precursors, ZIF-8P-PolybHb NPs exhibited significantly enhanced stability against H₂O₂ in comparison to both cell-free bHb and PolybHb. In FIG. 10B, only 10.25±1.20%, 14.60±1.12%, and 19.81±0.34% of ZIF-8P-PolybHb was oxidized to hemichrome (3.52±1.79%, 3.34±6.71%, and 6.02±0.33%) and metHb (6.70±0.56%, 7.80±2.20%, and 13.69±0.66%) at a Hb concentration of 0.5, 0.25, and 0.125 mg/mL, respectively. Overall, the activity of the bHb cross-linked into PolybHb was better preserved, when we encapsulated PolybHb using ZIF-8 precursors, demonstrating enhanced protection against H₂O₂ oxidation versus encapsulated PolybHb.

### PolybHb Encapsulation Efficiency (EE %) and Hb Loading.

The overall Hb encapsulation efficiency (EE %) of ZIF-8P-PolybHb NPs was calculated based on the total mass of encapsulated PolybHb and the initial mass of PolybHb. ZIF-8P-PolybHb NPs were observed to exhibit an ultrahigh Hb encapsulation efficiency (EE %=˜93.0±3.8%) compared to ZIF-8P-Hb NPs (EE %=˜88.2±3.5%). The higher Hb encapsulation efficiency could be the result of chelation mediated chemical etching of the ZIF-8P-PolybHb NPs, which generate larger cavities that enable higher protein loading capacity. Additionally, the Hb content of ZIF-8P-PolybHb NPs (4.65±0.02 mg/mL) was found to be slightly higher than that of ZIF-8P-Hb NPs (4.41±0.18 mg/mL). ZIF-8P-PolybHb NPs were also observed to have a larger hydrodynamic diameter in comparison to ZIF-8-Hb NPs, which could also result in a larger pore volume.

PolyHb Leakage from ZIF-8P-PolybHb NPs.

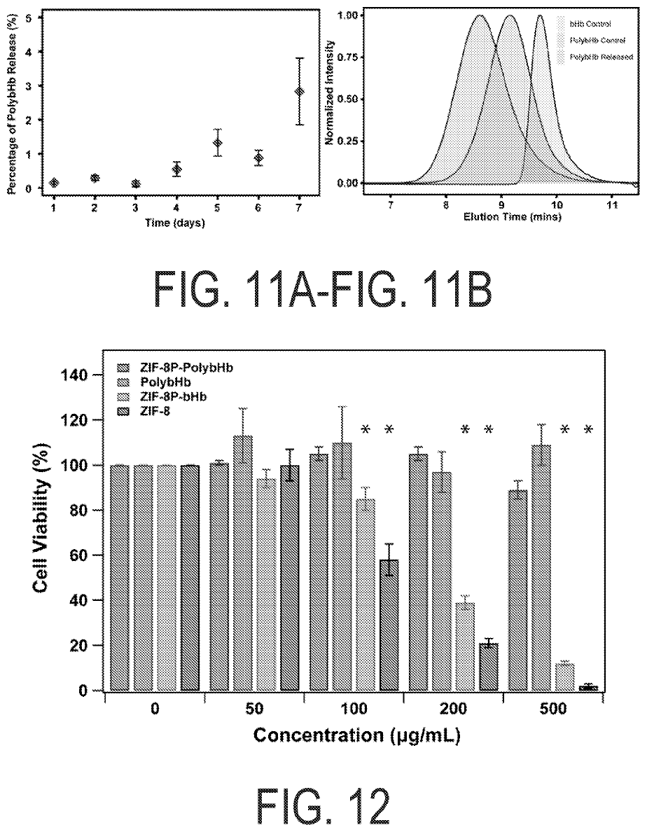

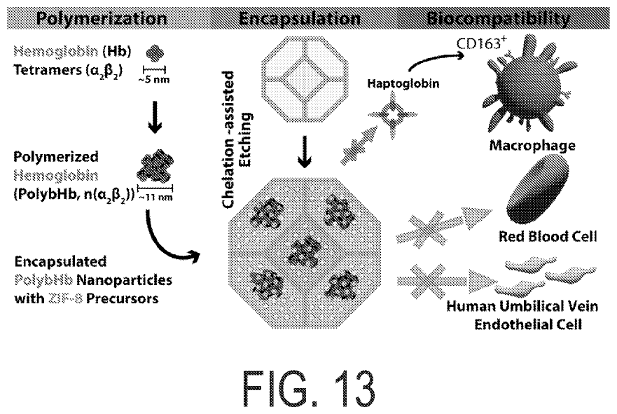

In FIG. 11A, ZIF-8P-PolybHb NPs were observed to gradually release PolybHb from 0.15±0.07% to 2.83±0.97% from day 1 to 7 at 37° C., demonstrating favorable long term hydrothermal stability in aqueous solution. The released PolybHb was quantified via SEC-HPLC as shown in FIG. 11B. The majority of PolybHb was retained inside ZIF-8P-PolybHb NPs, suggesting that the modified pores/cavities in the NPs only facilitate the transport of low MW PolybHb through the NP pores. Based upon the results above, we can conclude that ZIF-8P-PolybHb NPs mitigate ROS generation despite the formation of metPolybHb at physiological temperatures due to the relatively low PolybHb leakage from the ZIF-8P-PolybHb NPs.

### Hemocompatibility.

The interaction between ZIF-8P-PolybHb NPs and RBCs was studied by monitoring the hemolytic activity of the mixture. During hemolysis, Hb tetramers which are composed of two αβ dimers tend to dissociate at relatively low Hb concentration, which can be excreted through the kidney and eventually cause renal failure. Thus, it is important to measure the effect of ZIF-8P-PolybHb NPs on RBC hemolysis. Briefly, ZIF-8P-PolybHb NPs exhibited relatively low hemolysis (˜3.7%), demonstrating favorable hemocompatibility (<5% hemolysis) according to the ASTM E2524-08 standard.

### Encapsulation of PolybHb Reduces the Cytotoxicity of ZIF-8 NPs.

The in vitro cytotoxicity of ZIF-8P-PolybHb NPs compared to unloaded and bHb loaded ZIF-8 NPs was tested in HUVECs and presented below in FIG. 12. At concentrations up to 500 μg/mL, ZIF-8P-PolybHb NPs showed no statistically significant reduction in cell viability compared to a positive control of untreated cells, maintaining cell viability of up to 89%+9%. In addition, PolybHb by itself had no statistically significant impact on cell viability at all measured concentrations compared to the positive control (p=0.9976). Comparatively, ZIF-8P-PolybHb NPs showed improved cytotoxicity compared to unloaded and bHb loaded ZIF-8 NPs at concentrations of 100 μg/mL and above. At a concentration of 100 μg/mL, ZIF-8 NPs maintained 58±15% cell viability, whereas ZIF-8P-PolybHb NPs at that concentration maintained comparable cell viability to the positive control (p<0.0001). Cell viability further decreased with increasing concentration of ZIF-8 NPs causing almost complete cell death of HUVECs at 500 μg/mL compared to the positive control (p<0.0001), showing a continued significant difference between ZIF-8 NPs and ZIF-8P-PolybHb NPs at 200 μg/mL and 500 μg/mL (p<0.0001), validating the theory that encapsulation of PolybHb into the ZIF-8 NP structure markedly improves the cytotoxicity of ZIF-8 NPs. Encapsulation of bHb into the ZIF-8 NP nanostructure, improves the cytotoxicity compared to unloaded ZIF-8 NPs at 100 g/mL and 200 μg/mL (p<0.0001), but no significant difference was found comparing unloaded and bHb loaded ZIF-8 NPs impact on cell viability at 500 μg/mL (p=0.1176). Directly comparing the impact of PolybHb encapsulation to non-polymerized bHb encapsulation in improving the cytotoxicity of ZIF-8 NPs, a significant difference was measured at concentrations of 200 μg/mL and 500 μg/mL (p<0.0001), while no significant difference in cell viability was found between the two treatments at 100 μg/mL (p=0.2006), further strengthening the case for ZIF-8P-PolybHb NPs as a novel biocompatible HBOC.

### CONCLUSION

In this Example, we successfully synthesized encapsulated PolybHb into NPs using ZIF-8 precursors (ZIF-8P-PolybHb). Additionally, we established and optimized a continuous-injection method coupled with a chelation-assisted chemical etching process to synthesize ZIF-8P-PolybHb NPs. ZIF-8P-PolybHb NPs were <300 nm in diameter and exhibited O₂ binding properties similar to that of unencapsulated PolybHb. ZIF-8P-PolybHb NPs also exhibited favorable antioxidant (H₂O₂) properties and hemocompatibility, demonstrating its potential as an RBC substitute. ZIF-8P-PolybHb showed no statistically significant impact on HUVEC viability at all concentrations and encapsulation of PolybHb into the ZIF-8 framework improved cell viability compared to unloaded ZIF-8 NPs and bHb loaded ZIF-8 NPs

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the methods described herein without departing from the concept, spirit and scope of the disclosure. More specifically, it will be apparent that certain agents which are both chemically related may be substituted for the agents described herein while the same or similar results would be achieved. All such similar substitutes and modifications apparent to those skilled in the art are deemed to be within the spirit, scope and concept of the disclosure as defined by the appended claims.

1. A method for producing a population of matrix-encapsulated protein particles, the method comprising:
(a) combining a first framework precursor, a second framework precursor, and a protein to form a reactant mixture;
(b) incubating the reactant mixture under conditions effective to form the population of matrix-encapsulated protein particles; and
(c) separating the matrix-encapsulated protein from the reactant mixture using ultrafiltration;
wherein the matrix-encapsulated protein particles comprise a protein encapsulated in a porous framework formed by reaction of the first framework precursor and the second framework precursor.

2. The method of claim 1, wherein the population of matrix-encapsulated protein particles has an average particle size, as determined by electron microscopy, of less than 200 nm, such as less than 180 nm, less than 160 nm, less than 140 nm, less than 120 nm, less than 100 nm, or less than 80 nm; or wherein the population of matrix-encapsulated particles has an average particle size of at least 500 nm, at least 750 nm, at least 1 micron, at least 1.5 microns, at least 2 microns, at least 2.5 microns, at least 5 microns, at least 10 microns, at least 20 microns, at least 30 microns, at least 40 microns, at least 50 microns, or at least 100 microns.

3. The method of claim 1, wherein the method exhibits an encapsulation efficiency, measured by the fraction of the mass of the protein in the resulting matrix-encapsulated protein over the total mass of protein initially charged in the reactant mixture, of at least 80%, such as at least 82%, at least 84%, at least 86%, at least 88%, at least 90%, at least 92%, at least 94%, or at least 96%.

4. The method of claim 1, wherein the matrix-encapsulated protein particles have a zeta potential of less than −5 mV, such as of less than −6 mV, less than −7 mV, less than −8 mV, less than −9 mV, less than −10 mV, less than −11 mV, less than −12 mV, less than −13 mV, less than −14 mV, or less than −15 mV.

5. The method of claim 1, wherein the population of particles has a PDI of less than 0.100, such as less than 0.095, such as less than 0.090, less than 0.085, less than 0.080, less than 0.075, or less than 0.070.

6. The method of claim 1, wherein the porous framework comprises a metal-organic framework (MOF), metal-inorganic framework (MIF), and/or covalent-organic framework (COF).

7. (canceled)

8. The method of claim 1, wherein the first framework precursor comprises a metal salt and the second framework precursor comprises a ligand.

9. The method of claim 1, wherein the porous framework comprises a zeolitic imidazolate framework (ZIF).

10. (canceled)

11. The method of claim 1, wherein the first framework precursor comprises a Fe salt, a Co salt, a Cu salt, a Zn salt, or a combination thereof.

12. The method of claim 1, wherein the second precursor comprises a ligand selected from the group consisting of imidazoles and derivatives such as 2-methylimidazole, 2-ethylimidazole, 4-azabenzimidazole, benzimidazole, nitroimidazole, 2-chloroimidazole, and the like; carboxylic acids and derivatives such as 1,4-benzenedicarboxylic acid, 1,3,5-benzene tricarboxylic acid, imidazole carboxaldehyde, 2-aminobenzimidazolate, the like, or any combination thereof.

13. The method of claim 1, wherein the first framework precursor and the second framework precursor are present in the reactant mixture at a molar ratio of from 1:1 to 75:1, such as from 1:1 to 60:1, from 1:1 to 30:1, or from 15:1 to 30:1.

14. The method of claim 1, wherein the protein is selected from the group consisting of conalbumin, albumin, hemoglobin, haptoglobin, hemopexin, transferrin, methemoglobin, ovalbumin, α-chymotrypsinogen A, α-chymotrypsin, trypsin, trypsinogen, β-lactoglobulin, myoglobin, α-lactalbumin, lysozyme, ribonuclease A, or cytochrome c, a recombinant version thereof, or a combination thereof.

15. (canceled)

16. (canceled)

17. (canceled)

18. (canceled)

19. (canceled)

20. The method of claim 1, wherein the reactant mixture further comprises an etching agent, a chelating agent, or a combination thereof.

21. The method of claim 20, wherein the etching agent is comprises hydrofluoric acid (HF), ammonium fluoride (NH₄F), the acid salt of ammonium fluoride (NH₄HF₂), sodium hydroxide (NaOH), nitric acid (HNO₃), hydrochloric acid (HCl), hydroiodic acid (HI), hydrobromic acid (HBr), boron trifluoride (BF₃), sulfuric acid (H₂SO₄), acetic acid (CH₃COOH), formic acid (HCOOH), phosphoric acid (H₃PO₄), or any combination thereof.

22. The method of claim 1, wherein the chelating agent comprises ethylenediaminetetraacetic acid (EDTA) or a derivative thereof.

23. The method of claim 1, where step (c) comprises filtering the reactant mixture comprising the matrix-encapsulated protein particles by ultrafiltration against a filtration membrane, thereby forming a retentate fraction comprising matrix-encapsulated protein particles having a molecular weight above a cutoff value and a permeate fraction comprising unencapsulated protein and other impurities having a molecular weight of less than the cutoff value.

24. (canceled)

25. (canceled)

26. (canceled)

27. A pharmaceutical composition comprising a population of matrix-encapsulated hemoglobin particles prepared using the method defined by claim 1.

28. A method of treating hypoxia in a subject comprising administering the subject a therapeutically effective amount of the composition defined by claim 27.

29. (canceled)

30. A method for producing a population of matrix-encapsulated protein particles, wherein the protein comprises methemoglobin or polymerized methemoglobin, the method comprising:
(a) combining a first framework precursor, a second framework precursor, and hemoglobin or polymerized hemoglobin to form a reactant mixture;
(b) incubating the reactant mixture under conditions effective to form the population of matrix-encapsulated protein particles;
(c) contacting the population of matrix-encapsulated protein particles with an oxidizing agent under conditions effective to convert the hemoglobin or polymerized hemoglobin to methemoglobin or polymerized methemoglobin; and
(d) separating the matrix-encapsulated protein from the reactant mixture using ultrafiltration;
wherein the matrix-encapsulated protein particles comprise a protein encapsulated in a porous framework formed by reaction of the first framework precursor and the second framework precursor.

31-34. (canceled)

35. A method for producing a population of matrix-encapsulated protein particles, wherein the protein comprises methemoglobin or polymerized methemoglobin, the method comprising:
(a) contacting hemoglobin or polymerized hemoglobin with an oxidizing agent under conditions effective to convert the hemoglobin or the polymerized hemoglobin to methemoglobin or polymerized methemoglobin;
(b) combining a first framework precursor, a second framework precursor, and the methemoglobin or the polymerized methemoglobin to form a reactant mixture;
(c) incubating the reactant mixture under conditions effective to form the population of matrix-encapsulated protein particles; and
(d) separating the matrix-encapsulated protein from the reactant mixture using ultrafiltration;
wherein the matrix-encapsulated protein particles comprise methemoglobin or polymerized methemoglobin encapsulated in a porous framework formed by reaction of the first framework precursor and the second framework precursor.

36-38. (canceled)
